(19) United States
(12) Patent Application Publication
(10) Pub. No.: US 2026/0283888 A1
(43) Pub. Date: Sep. 24, 2026

(54) MOTORIZED STATIONARY EXERCISE APPARATUS

(71) Applicants: Fred M. Discenzo, Brecksville, OH (US); Kenneth A. Loparo, Chesterland, OH (US); Angela L. Ridgel, Rocky River, OH (US); Haston Abdar, Mayfield Heights, OH (US)

(72) Inventor: Fred M. Discenzo, Brecksville, OH (US)

(21) Appl. No.: 19/567,163

(22) Filed: Mar. 15, 2026

Related U.S. Application Data
(60) Provisional application No. 63/775,995, filed on Mar. 21, 2025.

(51) Int. Cl. A61H 1/02; G16H 20/30

(52) U.S. Cl. CPC A61H 1/0214; A61H 2201/1215; A61H 2201/5007; A61H 2201/5058; A61H 2201/5097; G16H 20/30

(57) ABSTRACT

A motorized stationary exercise apparatus includes a motor-driven pedal assembly, at least one sensor configured to measure rider interaction, a personalized exercise planning system, and a control system configured to dynamically personalize operation of the apparatus. The control system generates and continuously updates a rider-specific adaptive performance model that predicts future rider performance. Target motor operating parameters for each exercise session are determined by the personalized exercise planning system to optimize a rider performance objective function, and the electric motor dynamically adjusts pedal speed and torque during and across exercise sessions. The system may implement model-based predictive control, model-free estimation including artificial neural networks, or reinforcement learning or extremum seeking control. The adaptive model may estimate latent rider state variables including fatigue, endurance, strength, or neuromotor capability. The apparatus supports rehabilitation, neurological therapy, performance training, neuromotor entrainment, remote monitoring, multi-device coordination while incorporating safety detection and automatic torque reduction mechanisms.

### BRIEF SUMMARY

The present invention relates to a motorized stationary exercise apparatus having a motor-driven pedal assembly and adaptive planning and control architectures that are configured to dynamically personalize exercise planning and operation for an individual rider.

The apparatus includes a frame supporting a seat and handlebars, a pedal assembly mechanically coupled to an electric motor, at least one sensor configured to measure rider interaction with the pedal assembly, a planning system that personalizes exercise routines, and a control system operatively coupled to the motor and the sensors. The planning system provides personalized setpoints for each exercise session and the control system simultaneously manages cadence and torque based on the setpoints and generates and continuously updates a rider-specific adaptive performance model representing a current performance state of the rider.

Using the adaptive planning and performance model, the system predicts future rider performance under alternative pedal speed (cadence) and torque conditions and determines target motor operating parameters that optimize a rider performance objective function subject to safety constraints. The planning system generates setpoints for an exercise session and the electric motor is dynamically controlled to adjust pedal speed and torque during an exercise session and across multiple sessions, thereby personalizing therapy or performance training for the rider.

In various embodiments, the exercise planning system may implement forward and inverse predictive models using model-based techniques, and model-free techniques using statistical data-driven and AI/ML methods. The system may include static models based on population data, or learning methods that dynamically adjust the static model after each exercise session to personalize and adapt the model to the unique characteristics of a rider.

In various embodiments, the control system may implement adaptive model-based predictive control, model-free estimation using a trained data-driven predictor such as an artificial neural network, or extremum seeking control to optimize rider performance. The system may estimate latent rider state variables including fatigue level, endurance capacity, strength capacity, and neuromotor stability.

The apparatus may further include safety monitoring configured to detect abnormal operating conditions and automatically reduce speed and/or motor torque, as well as a communications module enabling remote monitoring and modification of therapy parameters by clinical personnel or a trainer.

The invention is particularly suited for therapeutic applications, including rehabilitation and management of neurological conditions such as Parkinson's disease, while also being applicable to general fitness and performance enhancement.

### BRIEF DESCRIPTION OF THE FIGURES

FIG. 1 is a graphic illustration of an example motorized stationary exercise apparatus, according to some embodiments of the present disclosure.

FIG. 2 is a graphic illustration of an example stationary exercise apparatus showing the location of certain electrical and electro-mechanical components according to some embodiments of the present disclosure.

FIG. 3 is a graph showing a representative example of the trajectory of rider performance management in an exercise session.

FIG. 4 is a graph showing a representative example of the trajectory of rider performance management over a sequence of exercise sessions.

FIG. 5 is a block diagram illustrating functional elements for adaptive dynamic exercise control in an exercise session.

FIG. 6 is a block diagram illustrating the functional elements for adaptive dynamic exercise control over a sequence of exercise sessions.

FIG. 7 is an illustration of a two-person motorized stationary exercise apparatus, according to some embodiments of the present disclosure.

FIG. 8 is a block diagram illustrating the functional elements for dynamic exercise control for a two-person motorized stationary exercise apparatus, according to some embodiments of the present disclosure.

FIG. 9 is a block diagram illustrating the sequence of steps used to optimize rider performance using an adaptive rider model, according to some embodiments of the present disclosure.

FIG. 10 is a graphic representation of a conceptual example of neuronal entrainment.

FIG. 11 is a block diagram illustrating an exemplary computer system able to operate the exercise apparatus.

FIG. 12 is a graphic representation of a client-server communication system.

### DETAILED DESCRIPTION

A stationary exercise device that can safely assist a person in performing movement in order to improve physical abilities in an optimal or near-optimal manner is described. Core elements include a motor-driven exercise device coupled with an adaptive rider model and an exercise planning module that directs motor control in a manner that will achieve rider-specific performance objectives for single and across and across multiple exercise regimens.

Various aspects are now described with reference to the drawings. In the following description, for purposes of explanation, numerous specific details are set forth in order to provide a thorough understanding of one or more aspects. It may be evident, however, that such aspect(s) may be practiced without these specific details. In other instances, well-known structures and devices are shown in illustrative and block diagram form in order to facilitate describing these aspects.

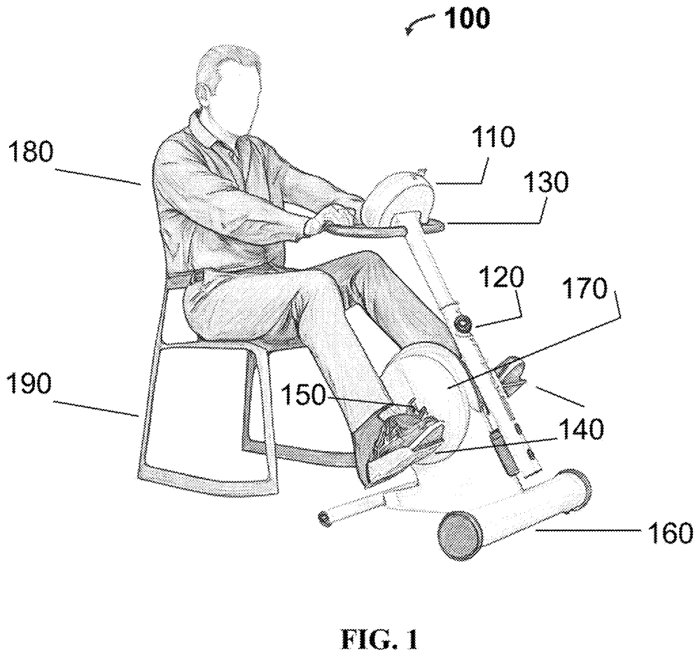

Turning now to the figures, FIG. 1 is an illustration of an example motor-driven exercise device 100 that includes a rider control and display component 110 mounted to the exercise device frame 120 permitting a rider to view details about the operation of the exercise device and details about the current exercise session. Control and display component 110 permits a rider or trainer to change exercise parameters, save exercise data and communicate bidirectionally via wired or wireless communications with an external entity. Additionally, hand grip heart rate monitors (not shown) may be integrated with the handlebars 130 and the external seat 190. Foot pedals 140 that each travel in a circular (or elliptical or linear) path are connected to a foot pedal crank 150 that connects to an electric motor (not shown). A secure floor mounting 160 integrated with the exercise frame 120 helps maintain the stability of the exercise device during operation and prevents the device from tipping or falling. A cover or shroud 170 housing the motor, drive, electronics and mechanical drive mechanism is shown that protects the interior components and prevents injury to the rider. In this illustration, the rider 180 will sit in a chair 190 that is not integral to the exercise device. Chair 190 is located in close proximity to the pedals 140 with both feet on the pedals 140 and holding handlebars 130 to help maintain balance while operating the exercise device. The chair 190 is located at an appropriate distance from the pedals for each rider to ensure optimal knee angle for efficient, injury-free pedaling. Optionally, the chair 190 may be mechanically attached to the exercise device frame 120 and may include a reclining seat or arm rests or side grips to enhance rider safety. Additionally, the chair 190 may be replaced by a wheel chair for the exercise device to accommodate a person with limited mobility.

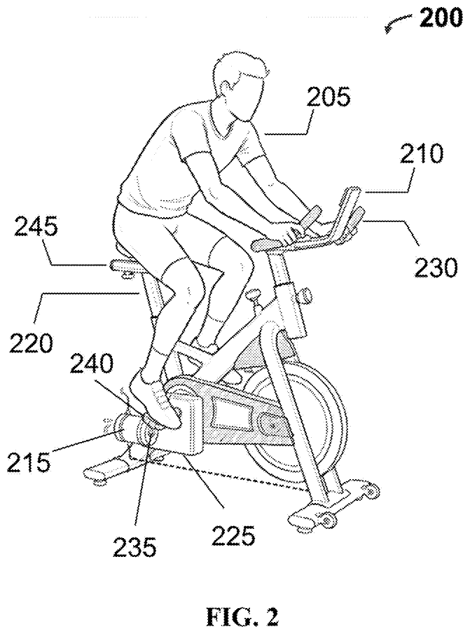

FIG. 2 is an illustration of an example exercise device 200 showing a rider 205 seated on an exercise device seat 245 attached to frame 220 with feet attached to the pedals 240 and holding handlebars 230 while viewing rider control and display component 210. The foot pedals 240 are mechanically coupled to electric motor 215 through a pedal crank (not shown). The electric motor 215 is controlled by motor controller 225. The electric motor provides power to assist the rider in pedaling the exercise device or provides pedal resistance to cause the rider to exert greater energy to achieve a target pedal cadence. Motor operation may dynamically switch between providing power to assist in pedal movement and providing braking power requiring increased power required from the rider to achieve a target pedal cadence. The operation of the motor is managed by motor controller 225. In some embodiments, the motor control system 225 may include a processor, memory for storing executable instructions including software for operating the motor, power electronics for managing power to the motor and software for managing the rider display 210, managing data storage and data communications, a model of the rider and personalized planning logic for prescribing the future operation of the exercise device. The motor controller 225 dynamically adjusts speed and torque applied to the pedals 240 in accordance with the target motor operating parameters during an exercise session and across a plurality of exercise sessions. In some embodiments, the control system (not shown) embedded in motor controller 225 generates and updates a rider-specific adaptive performance model representing a current performance state of the rider based on the signals representative of rider 205 interactions with the pedal assembly 240. Additionally, information from the rider during operation such as from handlebar 230 mounted contact heart rate sensors may be used along with exercise device-mounted sensors 235 such as a motor speed sensor and rider power measurements may be used to ensure accurate and timely updating of the rider-specific performance model. In some embodiments, the rider-specific model may predict a future performance state of the rider under one or more prospective operating conditions of the pedal assembly 240 and use this information to prescribe a change in motor 215 operation that will optimize a rider performance objective function.

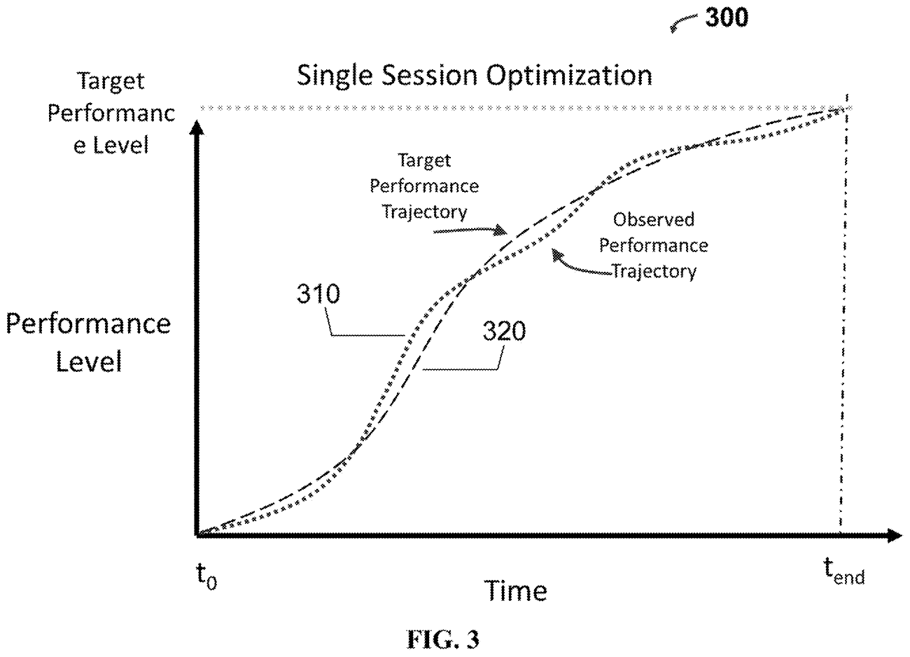

FIG. 3 illustrates an example of rider performance tracking and optimization during operation of an exercise device beginning at time to and ending at time tend. The observed performance level of the rider 310 is tracked using motor and motor drive information and optionally additional information from device-mounted and rider-mounted sensors. The performance metric may be a combination of rider strength and ability values or may be an objective function that combines multiple observable rider performance parameters. Rider observed performance is continually compared with the nominal or desired performance target level 320 throughout operation of the exercise device until the end of the exercise session at time tend. Deviation between the observed performance level and the target performance level may cause a change in the motor controller to assist the rider reach the target performance level by the end of the exercise session tend. Continual feedback describing rider performance and the use of the adaptive rider model enables the system to dynamically “adapt” and “tune” the change in bike operating parameters needed to optimize rider benefit.

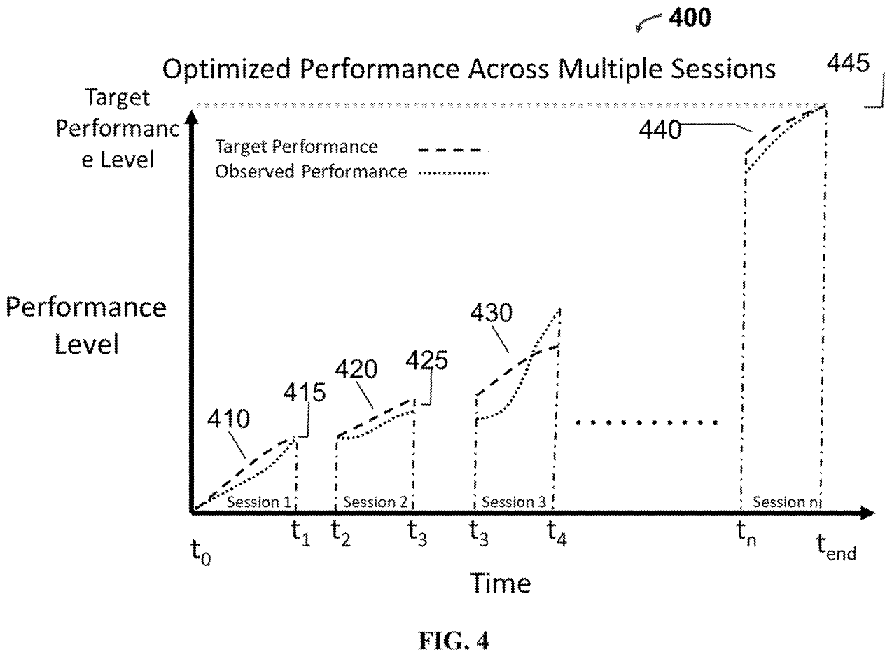

In FIG. 4 provides a graphical representation showing an example of how the scheduled rider performance level for individual exercise sessions 410 420 430 440 can be defined and managed to ensure that at the end time tend of multiple exercise sessions, the rider achieves an optimum, targeted level of performance. Each individual exercise session provides a target performance level and tracks the rider's performance and adjusts the controller to aid the rider in achieving the targeted performance level at the end of each individual performance session. At the end of each individual exercise session the performance of the rider may match the target performance level 415 or may be below the targeted performance level 425 or may be above the targeted performance level 435. Rider model adaptation from prior exercise sessions and the performance gap observed from the most recent exercise session will be used by the exercise planning module to instruct the motor controller to initiate control changes as needed at the start of the next exercise session and throughout the exercise session to keep the rider on track to achieve the end target performance level 445 at tend, when the sequence of separate exercise sessions conclude.

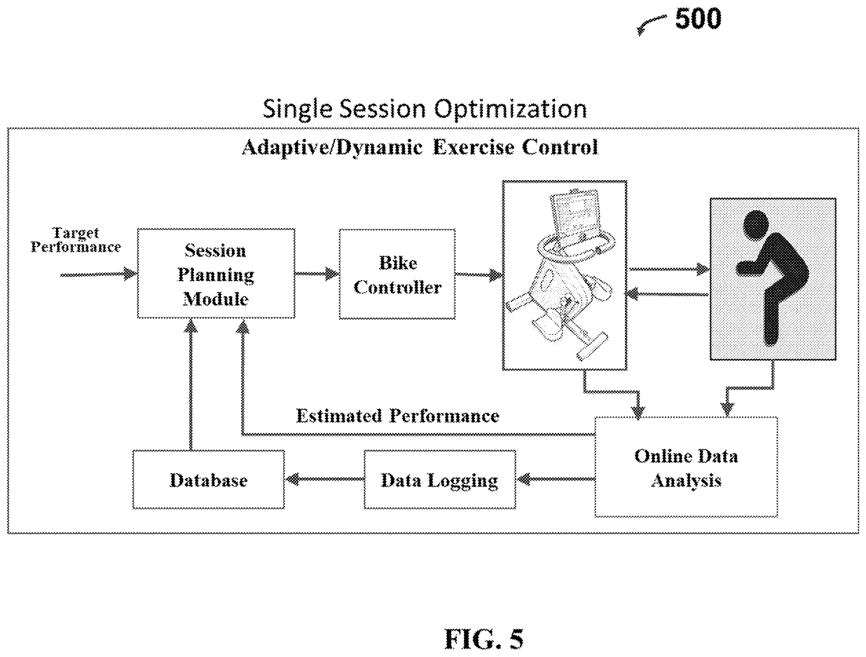

FIG. 5 provides a block diagram 500 showing a closed-loop exercise planning and adaptive control system that operates the motorized stationary exercise device in a manner to achieve a pre-defined target performance level of the rider during an exercise session. Data during operation from the rider and from device-mounted sensors are captured, logged and analyzed. The analysis can include an assessment of the condition of the device and an estimate of the rider's performance level, as well as other analysis methods. Real-time performance estimates coupled with historical database information is used by the exercise session planning module to determine what changes, if any, are needed in an exercise session in order for the rider to achieve safety goals and meet the target performance level at the end of an exercise session. Integral to the exercise session planning module is an adaptive model of the rider. This model is updated based on the rider's stimulus response observed and historical exercise information. The model is used to estimate the future performance of the rider to potential future control actions and control strategies. A control action and control strategy providing the optimum safe benefit for the rider is selected. The resultant control action is sent to the Bike Controller to initiate prescribed changes in motor operation. An inner loop within the Bike Controller operates continuously as a feedback controller to ensure the motor-operated device provides consistent speed and torque in spite of load disturbances introduced by the rider or other external disturbances. The performance of the rider is continually estimated and compared with the desired or target performance level during exercise session 300 and changes in device operation are quickly defined and initiated by the Session Planning Center logic.

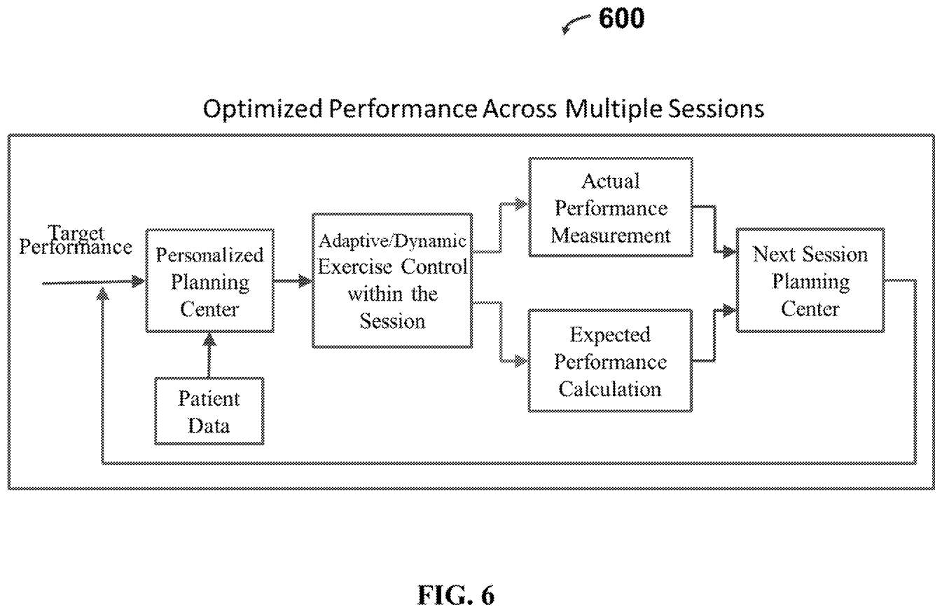

Similarly, FIG. 6 provides a block diagram 600 showing a closed-loop system that operates the motorized stationary exercise device in a manner to achieve a pre-defined target performance level after a sequence of individual performance sessions 400. A closed-loop system defines the target performance level of the rider based on rider data, a rider-specific model of rider response and differences between expected or desired rider performance and observed rider performance. The rider model integrated with the Personalized Planning Center module implements adaptive model-based predictive control to prescribe device operating conditions for the next riding session. Additionally, the rider-specific performance model is dynamically updated to improve accuracy for the next iteration of session planning with the objective of ensuring that at the end of the sequence of individual exercise sessions the rider will achieve an overall targeted level of performance.

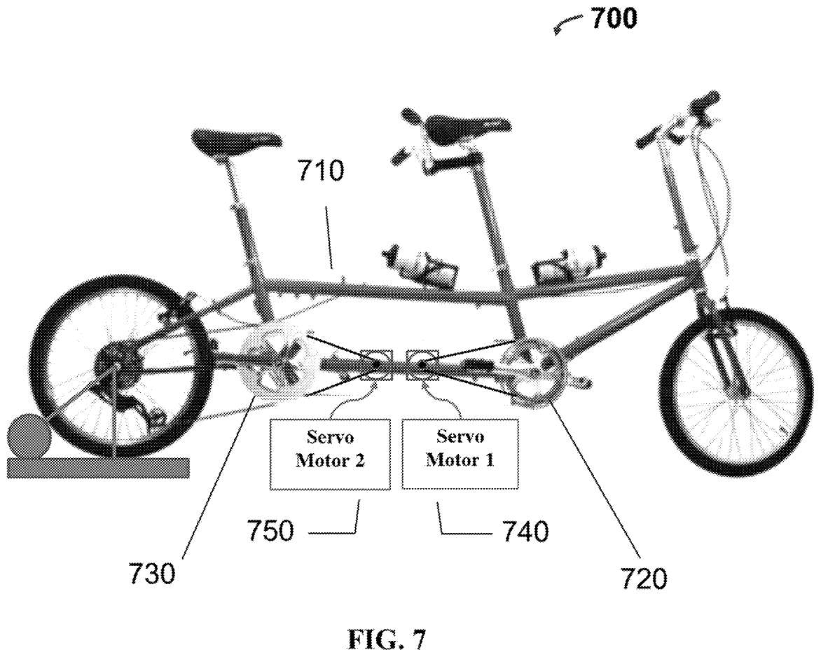

FIG. 7 presents a graphic showing a stationary tandem exercise bike with two seats, one for each rider, mounted on bike frame 710. Pedal cranks 720 730 with pedals, one set for each rider are each connected to separate servo motors 740 750. The pedal cranks 720 730 are each mounted to the bike frame 710 but the rotation of each pedal crank is independent from each other. Each pedal crank is controlled by independently connected servo motors 740 750. The tandem electro-mechanical system 700 provides the foundation enabling several exercise bike riders to ride computer-controlled exercise bikes concurrently and independently. More significant is the ability of the electro-mechanical control system to electronically couple the two pedal cranks for each rider to each other to achieve dynamic coordinated coupling of the motor controllers for each pedal crank.

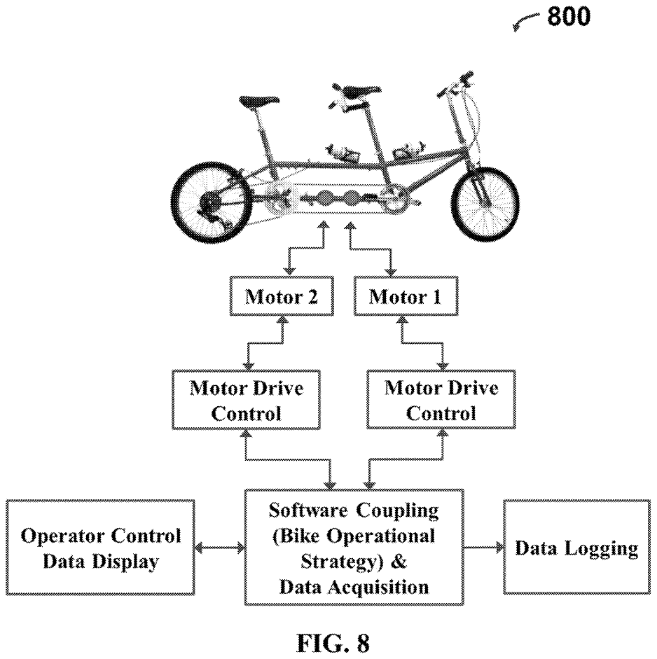

FIG. 8 is a graphic showing the independent motor drive for each servo motor and the coupling controller that provides the dynamic coordinated operation of both pedal cranks. The “stiffness” of the coupling of both pedal cranks is implemented in the Coupling Controller that coordinates the operation of each pedal crank servomotor concurrently. For example, pedal crank coupling strategy may prescribe a tight coupling between both pedal cranks causing both pedal cranks to operate and feel to the riders as if a rigid mechanical coupling exists between the two pedal cranks such as would be experienced with a sprocket chain mechanical coupling even though only an electronic, virtual crank coupling exists. In this case, slight disturbances from one rider would be immediately sensed by the other rider. Alternatively, a more compliant, softer, coupling may be implemented that gives each rider the feel of the other rider but as if the pedal cranks were connected with a compliant elastic belt. Still another opportunity exists for the coupling controller between both pedal cranks to filter out high frequency disturbances or torque fluctuations for one or both pedal crank servomotors.

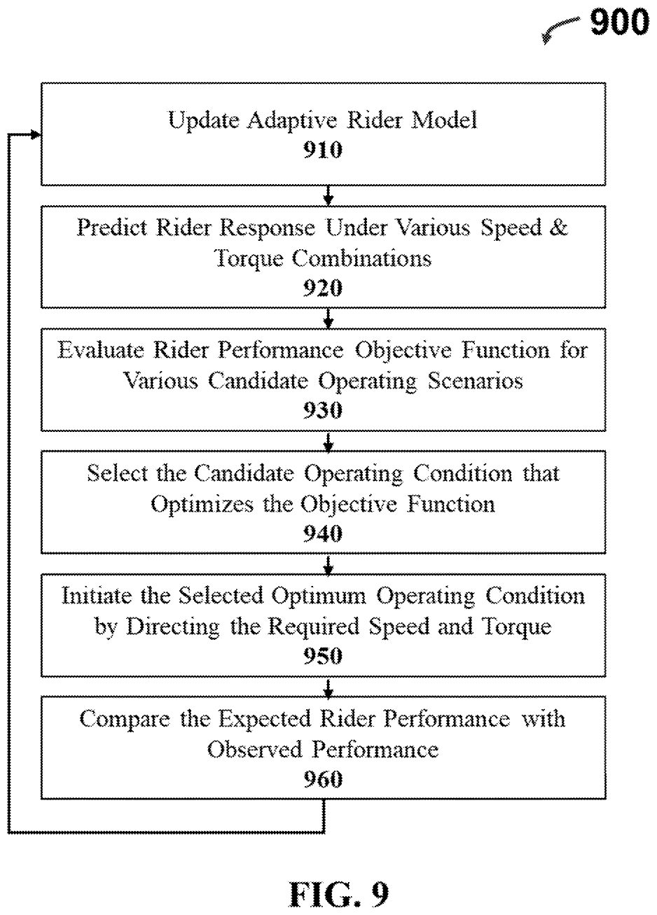

FIG. 9 shows a block diagram that describes the process of maintaining an adaptive rider module and using this module to select a bike operating condition that will optimize the rider's future performance. A closed-loop system is shown that continually updates the rider model to ensure an accurate and robust rider model and effective rider performance prediction. In particular, the adaptive rider module 910 is updated based on historical performance data and previous rider performance predictions. The improved rider model is then used to estimate the expected rider performance under a range of candidate motor-driven speed and torque combinations 920. The suite of potential riding outcomes generated is evaluated using a pre-defined objective function 940. The objective function defines the desired improvement in rider performance. In some embodiments the objective function may be comprised of an analytical representation of a combination of rider characteristics to be improved such as endurance, speed, rehabilitation progress, aerobic fitness, symmetric pedal torque or other therapeutic outcomes. Associated with the selected candidate motor-driven speed and torque combination is a series of commands sent to the motor controller to execute the specified control action 950. After initiating the specified control action 950, the actual rider performance is determined by analyzing motor drive operating conditions and optionally additional bike and rider sensor date. The observed rider performance is compared with the expected rider performance that was used to select the optimum control action 960. Any discrepancy between the predicted and expected rider performance values are used to update the adaptive rider model 910 as needed in a feedback loop.

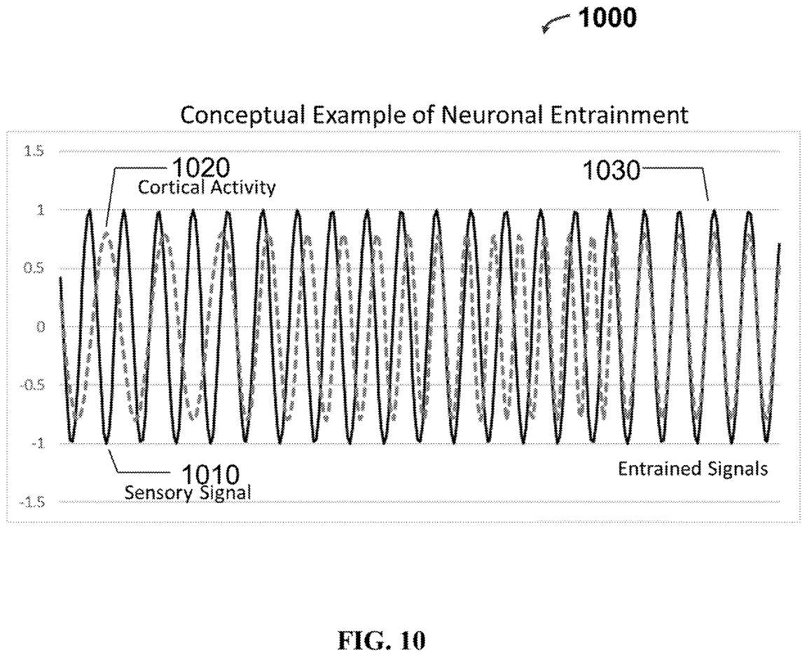

FIG. 10 provides a conceptual example of neuronal entrainment. External periodic stimuli such as from audio, visual, motion such as. dancing or cycling will result in afferent sensory signals 1010 reaching the cortex. Based on the frequency, consistency and intensity of the stimuli the sensory signals may cause a phase shift in the frequency of cortical activity 1020 causing the frequency of cortical activity to begin to align with the frequency of sensory signals 1030. Similar neuromotor entrainment may occur through dynamic speed and torque management during operation of the motor-controlled exercise device.

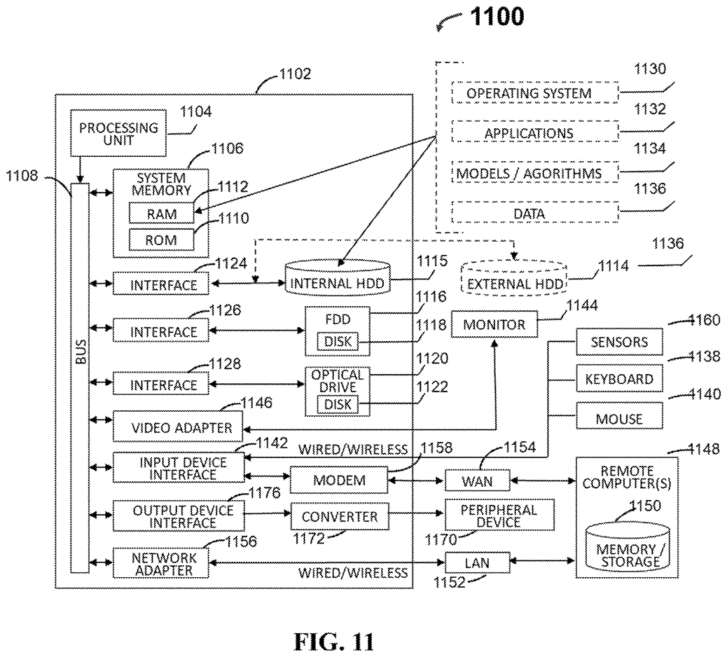

Referring now to FIG. 11, illustrated is a block diagram of a computer operable to execute the disclosed architecture. In order to provide additional context for various aspects of the subject invention, FIG. 11 and the following discussion are intended to provide a brief, general description of a suitable computing environment 1100 in which the various aspects of the invention can be implemented. While the invention has been described above in the general context of computer-executable instructions that may run on one or more computers, those skilled in the art will recognize that the invention also can be implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive systems and/or methods can be practiced with other computer system configurations, including single-processor or multiprocessor computer systems, minicomputers, mainframe computers, as well as personal computers, hand-held computing devices, microprocessor-based or programmable consumer electronics, single board computers, intelligent machines, distributed PLC's and the like, each of which can be operatively coupled to one or more associated devices. Coupling may be via wired (e.g. networked via Ethernet or DeviceNet) communications or wireless (e.g. RF, Bluetooth.™., IEEE 802.15.4, IEEE 802.11b, . . . ).

The illustrated aspects of the invention may also be practiced in distributed computing environments where certain tasks are performed by remote processing devices that are linked through a communications network. In a distributed computing environment, program modules can be located in both local and/or remote memory storage devices.

A computer typically includes a variety of computer-readable media. Computer-readable media can be any available media that can be accessed by the computer and includes both volatile and nonvolatile media, removable and non-removable media. By way of example, and not limitation, computer readable media can comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital video disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared, optical, and other wireless media. Combinations of the any of the above should also be included within the scope of computer-readable media.

With reference again to FIG. 11, the exemplary computing environment 1100 for implementing various aspects of the invention includes a computer 1102, the computer 1102 including a processing unit 1104, a system memory 1106 and a system bus 1108. The system bus 1108 couples system components including, but not limited to, the system memory 1106 to the processing unit 1104. The processing unit 1104 can be any of various commercially available processors. Dual microprocessors and other multi-processor architectures may also be employed as the processing unit 1104.

The system bus 1108 can be any of several types of bus structure that may further interconnect to a memory bus (with or without a memory controller), a peripheral bus, and a local bus using any of a variety of commercially available bus architectures. The system memory 1106 includes read only memory (ROM) 1110 and random access memory (RAM) 1112. A basic input/output system (BIOS) is stored in a non-volatile memory 1110 such as ROM, EPROM, EEPROM, which BIOS contains the basic routines that help to transfer information between elements within the computer 1102, such as during start-up. The RAM 1112 can also include a high-speed RAM such as static RAM for caching data.

The computer 1102 further includes an internal hard disk drive (HDD) 1115 (e.g., EIDE, SATA), which internal hard disk drive 1115 may also be configured for external use 1114 in a suitable chassis, a magnetic floppy disk drive (FDD) 1116, (e.g., to read from or write to a removable diskette 1118) and an optical disk drive 1120, (e.g., reading a CD-ROM disk 1122 or, to read from or write to other high capacity optical media such as the DVD). The hard disk drive 1115, magnetic disk drive 1116 and optical disk drive 1120 can be connected to the system bus 1108 by a hard disk drive interface 1124, a magnetic disk drive interface 1126 and an optical drive interface 1128, respectively. The interface 1124 for external drive implementations includes at least one or both of Universal Serial Bus (USB) and IEEE 1394 interface technologies. Other external drive connection technologies are within contemplation of the subject invention.

The drives and their associated computer-readable media provide nonvolatile storage of data, data structures, computer-executable instructions, and so forth. For the computer 1102, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods of the invention.

A number of program modules can be stored in the drives and RAM 1112, including an operating system 1130, one or more application programs 1132, system models and algorithms 1134 and program data 1136. All or portions of the operating system, applications, models, and/or data can also be cached in the RAM 1112. It is appreciated that the invention can be implemented with various commercially available operating systems or combinations of operating systems.

A user can enter commands and information into the computer 1102 through one or more wired/wireless input devices, e.g., a keyboard 1138, a pointing device, such as a mouse 1140. Information can also be received from sensors 1160. Other input devices (not shown) may include a microphone, an IR remote control, a joystick, console buttons or switches, pedals, a game pad, a stylus pen, touch screen, or the like. These and other input devices are often connected to the processing unit 1104 through an input device interface 1142 that is coupled to the system bus 1108, but can be connected by other interfaces, such as a parallel port, an IEEE 1394 serial port, a game port, a USB port, an IR interface, analog to digital (A/D) converters etc.

A monitor 1144 or other type of display device is also connected to the system bus 1108 via an interface, such as a video adapter 1146. In addition to the monitor 1144, a computer typically includes other peripheral output devices (not shown), such as speakers, printers, heads up displays, Augmented Reality goggles, etc.

Computer output may be used to directly communicate with external peripheral devices 1170 such as indicator lights, alarms, relays or contactors. This output may also direct other computer-based devices (e.g., motor-operated valve, variable frequency motor drive) to operate in a prescribed manner determined by processing unit 1104.

Processing unit 1104 utilizes applications 1132, models and algorithms 1134 and data 1136 to interpret external data received from input device interface 1142 and computes an output that will be transmitted to an external device using output device interface 1176. The computed output directs the operation of an external device as a result of the recent input received enabling the implementation of feedback control or closed-loop control using processing unit. The external devices controlled based on closed-loop computation by processing unit 1104 includes flow control, temperature control, speed control, torque control or chemical control for example. For example, closed-loop speed and torque control are integral to the operation of the motorized stationary exercise apparatus.

The computer 1102 may operate in a networked environment using logical connections via wired and/or wireless communications to one or more remote computers, such as a remote computer(s) 1148. The remote computer(s) 1148 can be a workstation, a server computer, a router, a personal computer, portable computer, microprocessor-based entertainment appliance, a peer device or other common network node, and typically includes any or all of the elements described relative to the computer 1102, although, for purposes of brevity, only a memory storage device 1150 is illustrated. The logical connections depicted include wired/wireless connectivity to a local area network (LAN) 1152 and/or larger networks, e.g., a wide area network (WAN) 1154. Such LAN and WAN networking environments are commonplace in offices, and companies, and facilitate enterprise-wide computer networks, such as intranets, all of which may connect to a global communication network, e.g., the Internet.

When used in a LAN networking environment, the computer 1102 is connected to the local network 1152 through a wired and/or wireless communication network interface or adapter 1156. The adaptor 1156 may facilitate wired or wireless communication to the LAN 1152, which may also include a wireless access point disposed thereon for communicating with the wireless adaptor 1156.

When used in a WAN networking environment, the computer 1102 can include a modem 1158, or is connected to a communications server on the WAN 1154, or has other means for establishing communications over the WAN 1154, such as by way of the Internet. The modem 1158, which can be internal or external and a wired or wireless device, is connected to the system bus 1108 via the input device interface 1142. In a networked environment, program modules depicted relative to the computer 1102, or portions thereof, can be stored in the remote memory/storage device 1150. It will be appreciated that the network connections shown are exemplary and other means of establishing a communications link between the computers can be used.

The computer 1102 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi, Bluetooth.™., IEEE 802.15.4, IEEE 802.11b, 802.11be, 802.11bn, and Zigbee™ wireless technologies. Thus, the communication can be a predefined structure as with a conventional network or simply an ad hoc communication between at least two devices.

Wi-Fi, or Wireless Fidelity, allows connection to the Internet from a couch at home, a hotel room, a coffee shop, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, be, bn etc.) to provide secure, reliable, fast wireless connectivity. A Wi-Fi network can be used to connect computers to each other, to the Internet, and to wired networks (which use IEEE 802.3 or Ethernet). Wi-Fi networks operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10BaseT wired Ethernet networks used in many offices. Each computer connected can serve as the host computer for one or more intelligent agents. The communications mechanism can be utilized to permit the agents to communicate with each other and collaborate to establish crane health, control strategy, re-configuration plans, and/or control plans. The network can serve to permit implementing both distributed and centralized control and permits remote sensors and actuators that are not directly wired to computer input channels to be accessed.

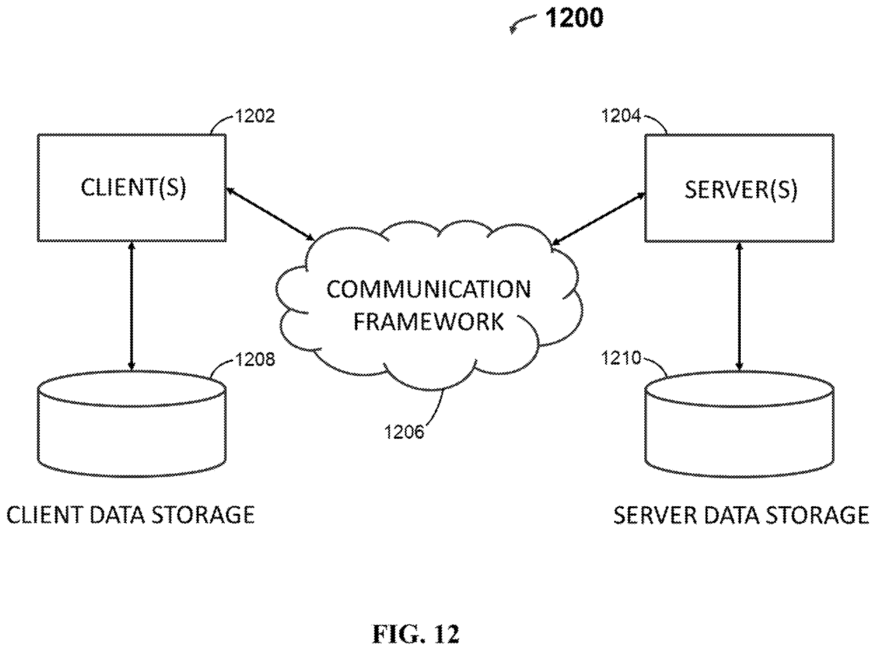

Referring now to FIG. 12, illustrated is a schematic block diagram of an exemplary computing environment 1200 in accordance with the subject invention. The system 1200 includes one or more client(s) 1202. The client(s) 1202 can be hardware and/or software (e.g., threads, processes, computing devices). The client(s) 1202 can house cookie(s) and/or associated contextual information by employing the invention, for example.

The system 1200 also includes one or more server(s) 1204. The server(s) 1204 can also be hardware and/or software (e.g., threads, processes, computing devices). The servers 1204 can house threads to perform transformations by employing the invention, for example. One possible communication between a client 1202 and a server 1204 can be in the form of a data packet adapted to be transmitted between two or more computer processes. The data packet may include a cookie and/or associated contextual information, for example. The system 1200 includes a communication framework 1206 (e.g., a global communication network such as the Internet) that can be employed to facilitate communications between the client(s) 1202 and the server(s) 1204.

Communications can be facilitated via a wired (including optical fiber) and/or wireless technology. The client(s) 1202 are operatively connected to one or more client data store(s) 1208 that can be employed to store information local to the client(s) 1202 (e.g., cookie(s) and/or associated contextual information). Similarly, the server(s) 1204 are operatively connected to one or more server data store(s) 1210 that can be employed to store information local to the servers 1204.

### BACKGROUND OF INVENTION

### Field of the Invention

The present invention relates generally to motorized stationary exercise planning and real-time equipment operation and, more particularly, to a motor-driven stationary exercise apparatus having a pedal assembly and a personalized exercise planning and adaptive control architecture configured to dynamically personalize motor operation based on a rider-specific performance model with the objective of maximizing the therapeutic and health benefits for the rider while ensuring rider safety.

### Description of Related Art

Stationary exercise bicycles are widely used in fitness training, rehabilitation, and therapeutic environments. Such devices are commonly deployed in homes, fitness centers, hospitals, and rehabilitation clinics. Many commercially available stationary exercise bicycles include electric motors that provide manual resistance or assistive torque adjustments to the pedal assembly. In some configurations, the motor may regulate pedal speed or provide resistance according to a pre-programmed exercise profile.

Conventional motorized exercise bicycles typically offer a selection of predefined exercise programs, resistance levels, or cadence targets. These programs are generally static in nature and are selected from a library of standardized exercise routines.

Although some systems allow manual adjustment of resistance or cadence during operation, the control architecture of such systems remains largely pre-programmed and non-adaptive.

Existing systems generally do not utilize a rider module and do not continuously update a rider-specific adaptive performance model. Instead, they rely on fixed parameters or limited real-time adjustments based solely on instantaneous measurements such as heart rate or cadence. These approaches fail to account for the significant variability among individual riders, including differences in strength, endurance, neuromotor control, disease progression, medication timing, fatigue levels, and recovery status.

In therapeutic applications, including treatment or management of neurological conditions such as Parkinson's disease, multiple sclerosis, or post-stroke rehabilitation, personalization of exercise parameters is particularly important. Riders with such conditions often exhibit variability in performance both within a single session and across multiple sessions. Static exercise programs may result in suboptimal therapeutic benefit, excessive fatigue, or even risk of injury.

Similarly, individuals recovering from orthopedic surgery, joint replacement, or other medical procedures may experience changing functional capacity over time. Conventional stationary exercise equipment does not dynamically adapt resistance and cadence targets based on predictive modeling of rider fatigue, strength, or endurance or recovery rate.

Some existing systems incorporate basic feedback control mechanisms that maintain a target cadence or resistance. However, these systems typically do not employ predictive control strategies, dynamic state estimation, model-based optimization, model-free learning techniques, or extremum seeking approaches to maximize individualized performance objectives.

Moreover, conventional stationary exercise bicycles lack integrated architectures that estimate latent rider state variables—such as fatigue rate, endurance capacity, or neuromotor performance—and use such estimates to optimize motor torque and speed in real-time. Without predictive and adaptive intelligence, these systems are unable to continuously refine exercise parameters in a given session or across multiple sessions to maximize therapeutic or performance outcomes for a specific individual.

In addition, remote monitoring capabilities in existing systems are often limited to passive data reporting. Few systems provide an integrated adaptive architecture that allows remote modification of optimization parameters while maintaining and continuously updating a rider-specific performance model.

Accordingly, there remains a need for a motorized stationary exercise apparatus that:
Continuously models rider-specific performance characteristics;
Predicts future rider performance under alternative operating conditions;
Dynamically optimizes pedal speed and torque using adaptive control techniques to optimize rider benefits;
Adjusts motor operation both within a session and across sessions;
Maximizes therapeutic and physical performance outcomes; and
Maintains safe operation through monitoring and automatic intervention.
   The present invention addresses these and other deficiencies in the art.

### SUMMARY OF THE INVENTION

The present invention provides a motorized stationary exercise apparatus having a motor-driven pedal assembly and an adaptive planning and control architecture configured to dynamically personalize an exercise regimen in concert with operation of the pedal assembly for an individual rider.

In one aspect, the invention comprises a stationary exercise apparatus including a frame supporting connection with an external seat and handgrips or an integrated seat and handlebars, a pedal assembly configured to be actuated by a rider, and an electric motor mechanically coupled to the pedal assembly such that the pedal assembly is motor-driven. At least one sensor generates signals representative of rider interaction with the pedal assembly, including pedal speed and pedal torque. A control system is operatively coupled to the motor and pedals and sensors.

The control system is integrated with a rider-specific model and continuously updates a rider-specific adaptive performance model representing the current performance state of the rider. The adaptive performance model may represent physiological, neuromotor, strength, endurance, or fatigue-related characteristics of the rider. The model is updated both during an exercise session and across multiple exercise sessions to reflect changes in rider capability and performance over time.

Using the adaptive performance model, the control system predicts future rider performance under one or more prospective pedal operating conditions, including alternative speed and torque combinations. The system evaluates a rider performance objective function associated with at least one of motor skill improvement, endurance improvement, strength improvement, aerobic capacity improvement, rehabilitation progress, or therapeutic outcome.

Target motor operating parameters are determined by optimizing the rider performance objective function. The electric motor is dynamically controlled to adjust speed and torque applied to the pedal assembly in accordance with the optimized target motor operating parameters. Adaptation occurs both within a single exercise session and across multiple sessions.

In certain embodiments, determining the target motor operating parameters includes adaptive model-based predictive control using the rider-specific adaptive performance model. In other embodiments, model-free estimation techniques may be used, including a trained data-driven predictor such as an artificial neural network established using techniques such as empirical risk minimization and configured to estimate rider's future response to changes in pedal speed and torque. In further embodiments, reinforcement learning or extremum seeking control may be employed to dynamically adjust control parameters to maximize the rider performance objective function.

In another aspect, the invention includes an adaptive rider module implemented by a processor and operatively coupled to the sensors and motor controller. The adaptive rider module constructs and continuously updates a rider-specific dynamic performance model, estimates latent rider state variables such as fatigue level or endurance capacity, predicts rider response under multiple prospective operating conditions, and determines optimized motor commands to improve individualized performance outcomes.

The apparatus may further include a user interface configured to display current and predicted rider performance metrics, as well as a communications module configured to transmit performance data to a remote system and receive modified operating parameters from a healthcare provider, therapist, or trainer.

Through dynamic modeling, prediction, and optimization of rider-specific performance characteristics, the invention provides a motorized stationary exercise apparatus capable of maximizing therapeutic and physical performance benefits while maintaining safe and adaptive operation.

### DETAILED DESCRIPTION OF ONE EMBODIMENT

In one embodiment, the invention comprises a motorized stationary exercise apparatus configured as a stationary exercise bicycle having a motor-driven pedal assembly and a control system configured to adapt operation of the apparatus for an individual rider. The apparatus includes an electric motor mechanically coupled to the pedal assembly and a control architecture configured to dynamically determine motor operating parameters based on measured rider interaction with the pedal assembly. In this embodiment, an adaptive rider module establishes and updates a rider-specific performance model representing a current performance state of the rider. The rider-specific performance model may include one or more variables indicative of rider fatigue, endurance capacity, neuromotor control stability, force production consistency, and energy expenditure rate. The control system uses the rider-specific performance model to determine motor speed commands, motor torque commands, or both, for controlling the electric motor during an exercise session.

During operation, rider-applied force to the pedal assembly changes the mechanical load seen by the motor and drivetrain. These load changes are detected, estimated, or inferred by the control system from sensor signals and motor operating parameters. The control system analyzes the load changes and other operating data to determine the rider's present operating condition and to predict rider response to prospective operating conditions. Rider's estimated response to prospective operating conditions is used to specify future motor control action.

### Mechanical Structure

The apparatus includes a frame configured to support a seat assembly, a hand-engagement assembly, and a pedal assembly. The frame may be configured as an upright bicycle frame, a recumbent bicycle frame, or another stationary cycling structure. The seat assembly and the hand-engagement assembly may be separately connected to the frame or may be integrated with the frame. The pedal assembly is rotationally supported by the frame and is configured to be actuated by a rider. In one embodiment, the pedal assembly includes left and right pedals, corresponding crank arms, and a crank axle or drive shaft. The pedal assembly may further include one or more bearings, housings, protective covers, and transmission components. An electric motor is mechanically coupled to the pedal assembly such that the electric motor can apply torque to the pedal assembly. The motor may apply assistive torque to increase pedal speed or reduce rider effort, or may apply resistive torque to oppose rider input. In some operating modes, the motor may alternate between assistive and resistive torque or may vary motor output as a function of rider state and exercise objectives.

In this embodiment, the electric motor is coupled to the pedal assembly through a gear reduction mechanism. The gear reduction mechanism may include one or more gears, sprockets, belts, chains, planetary stages, or equivalent torque-transmitting components. Other coupling mechanisms may also be used, including direct drive arrangements, belt drives, chain drives, or friction drives. The electric motor may comprise any suitable controllable motor, including a brushless DC motor, permanent magnet synchronous motor, AC motor, servo motor, or other rotary electric actuator. The motor may be selected to provide a desired continuous torque capability, peak torque capability, backdrivability, and thermal operating range.

Operation of the electric motor is managed by a motor controller configured to regulate electrical power delivered to the motor to achieve a target motor speed, a target motor torque, or both. The motor controller may comprise a variable frequency drive (VFD), inverter, servo drive, or other motor control circuitry. The motor controller may receive operating commands from a supervisory control system and may generate phase currents, switching signals, or other control outputs to cause the motor to operate at a commanded condition.

In one implementation, the motor controller is configured to maintain an established setpoint speed or torque, either of which may change dynamically during operation. A closed-loop feedback system is integrated with the motor controller to change power delivered to the motor in order to achieve and maintain the setpoint speed or torque. The motor controller may utilize sensor signals, estimated motor states, or both, to determine the operating state of the motorized exercise apparatus. In another implementation, motor operating information may be obtained from sensors integrated within the motor controller, such as a VFD, or is derived from drive operating parameters. Such information may include motor current, motor voltage, rotor speed, estimated torque, phase information, thermal information, or other controller-derived variables.

### Sensors

The apparatus includes a sensor subsystem configured to measure rider interaction with the pedal assembly and operation of the motorized drivetrain. The sensor subsystem may include discrete electrical, electromechanical, electro-optic, magnetic, or other sensors. Alternatively, or in addition, one or more sensed values may be derived from motor controller operating parameters or estimation algorithms.

In this embodiment, the sensor subsystem includes a torque or force sensor configured to measure pedal torque or force applied by the rider and/or the motor. The torque or force sensor may be located at the pedal, crank arm, crank axle, drive shaft, motor shaft, or another torque-transmitting member. In some embodiments, separate left-side and right-side force measurements may be obtained. The sensor subsystem further includes a rotational speed sensor configured to measure pedal cadence. The rotational speed sensor may comprise an encoder, tachometer, Hall-effect sensor, optical sensor, resolver, or equivalent rotational sensing device coupled to the crankset, drive shaft, flywheel, or motor. The sensor subsystem further includes a motor current sensor configured to measure electrical current associated with motor operation. Motor current measurements may be used to estimate motor torque contribution, motor loading, or power usage. In some implementations, motor torque contribution is estimated by algorithms embedded in the motor controller software.

Additional sensors may be included, such as pedal position sensors, crank angle sensors, motor temperature sensors, vibration sensors, heart rate sensors, oxygen saturation sensors, electromyography sensors, inertial sensors, acoustic sensors, optical position or feature sensors or other physiological or kinematic sensors. Signals from the sensor subsystem are provided to the control system for processing.

### Control System Architecture

The control system includes one or more processors and one or more memory devices storing executable instructions. The control system may be implemented in a centralized controller, in distributed controllers, or partially within the motor controller and partially in a supervisory processor.

In one implementation, the control system includes: (a) an adaptive rider module; (b) a motor controller configured to maintain target motor speed and torque values; and (c) an exercise planning module.

The motor controller may provide lower-level regulation of motor operation, while the adaptive rider module and the exercise planning module may provide higher-level determination of operating strategy, rider-specific adaptation, and session planning.

### Adaptive Rider Module

In this implementation, the adaptive rider module establishes and continuously updates a rider-specific dynamic performance model. The rider-specific dynamic performance model represents a current performance state of the rider and may include estimates of rider fatigue level, endurance capacity, neuromotor control stability, force production consistency, bilateral symmetry, response delay, and energy expenditure rate. The rider-specific dynamic performance model may be initialized during an early exercise session or during a setup and calibration procedure. During initialization, the apparatus may expose the rider to one or more known operating conditions and collect corresponding sensor data. Initial model parameters may be derived from the collected data.

The adaptive rider module refines the rider-specific dynamic performance model across subsequent sessions to improve predictive accuracy and to track changes in rider capability over time. The rider-specific dynamic performance model may therefore capture rider performance and exercise capacity, rider-specific riding characteristics, rider endurance, and rider response to motor assistance or resistance. The rider-specific dynamic performance model is used to predict future rider performance in response to potential changes in exercise bike control. In some embodiments, the model includes both short-term state variables that vary during a session and longer-term adaptation parameters that vary across sessions.

### Fatigue Estimation (Observer Implementation)

In one implementation, the adaptive rider module includes an observer configured to estimate latent model parameters, such as rider fatigue, that are not directly measured. The observer may comprise a state observer, Kalman filter, extended Kalman filter, recursive estimator, Bayesian estimator, or other estimation routine. The observer monitors dynamic deviations between predicted rider output and actual rider output under a given operating condition. For example, where cadence remains substantially constant and rider torque decreases, the observer may estimate an increase in rider fatigue. The observer may also evaluate torque variability, torque assymetry, cadence instability, delayed response to speed or torque changes, and deterioration in force symmetry.

In one implementation, the observer calculates: (a) an instantaneous fatigue estimate; (b) a rate of change of fatigue; and (c) a predicted future fatigue level under continued operating conditions. This information is used to adjust future pedal speed and torque commands to avoid overexertion while maintaining a desired exercise effect. For example, the control system may reduce resistive torque, increase assistive torque, reduce cadence, or otherwise modify operating parameters in response to an estimated increase in fatigue. Additionally, the rider's response to a step change, ramp change, or other perturbation in speed or torque may be used to characterize rider motor skill and to estimate rider fatigue level. Measured response characteristics may include response delay, overshoot, oscillation, recovery time, and symmetry of force generation.

### Model-Based Predictive Control

In one implementation, the control system implements adaptive model predictive control (MPC). At each control interval, the adaptive rider module predicts rider response under multiple prospective pedal speed and torque combinations using the rider-specific dynamic performance model. A rider performance objective function is evaluated for each candidate operating condition. The candidate operating condition that optimizes the objective function is selected, and the motor controller applies torque and speed commands corresponding to the selected operating strategy. The objective function may include weighted components representing one or more of:
(a) maintenance of target cadence;
(b) maximization of endurance time;
(c) minimization of fatigue rate;
(d) improvement of neuromotor performance;
(e) improvement in pedal force symmetry;
(f) reduction of cadence variability; and
(g) compliance with therapeutic or training constraints.

The MPC process repeats continuously during the riding session. In some embodiments, the optimization is subject to constraints including maximum torque, maximum cadence, maximum torque rate of change, maximum speed rate of change, thermal limits, and rider-specific safety limits.

### Alternative Control Strategy-Model-Free Estimation

In yet another implementation, the adaptive rider module includes a trained artificial neural network or other machine-learned predictive model. The predictive model is trained using historical rider performance data and is configured to predict rider response to changes in pedal speed and torque. During operation, the predictive model predicts rider performance for a plurality of candidate operating conditions. A decision module evaluates predicted outcomes, and the operating condition predicted to provide the best rider performance outcome is selected. The predictive model may be updated incrementally using new rider data collected during operation or after completion of one or more sessions.

### Alternative Control Strategy—Reinforcement Learning or Extremum Seeking Control

In an additional implementation, reinforcement learning, extremum seeking control, or another adaptive optimization strategy is used. The control system introduces perturbations in motor operating parameters and observes corresponding changes in a rider performance objective function. Based on measured gradients, reward signals, or other performance indicators, the control system adjusts motor commands in a direction expected to improve the objective function. This embodiment permits optimization without requiring an explicit analytical rider model.

### Within-Session and Cross-Session Adaptation

The adaptive rider module updates the rider-specific dynamic performance model continuously during a session and stores updated model parameters in memory.

During a subsequent session, the stored parameters are retrieved and used to guide selection of operating parameters for the current session. During the current session, the adaptive control system further refines the operating parameters based on newly acquired measurements. This provides adaptation both within a session and across multiple sessions. In some embodiments, the adaptation accounts for factors affecting rider performance, including medication timing, time-of-day variability, rehabilitation progress, disease progression, recovery state, and strength changes.

### Safety and Failsafe Operation

The control system monitors for abnormal operating conditions, including excessive torque spikes, sudden loss of torque, sudden spike in torque or cadence, cadence instability, torque assymetry, mechanical resistance anomalies, sensor disagreement, controller faults, and other unusual operating states. Upon detection of an unusual operating condition, the control system may classify the anomaly and determine an appropriate response. If an unknown or uncertain operating state is detected, the motor controller may transition immediately to a fail-safe operating state. The fail-safe operating state may include rapidly reducing motor torque, eliminating motor torque, transitioning to a neutral or backdrivable mode, applying controlled deceleration, or stopping motor operation. The apparatus may also provide a local or remote alert indicative of the detected condition. In some embodiments, different anomaly classifications correspond to different protective actions. Lower-severity conditions may result in warnings or reduced operating limits, while higher-severity conditions may result in immediate torque cutoff, motor-controlled dynamic braking or shutdown.

### User Interface and Remote Monitoring

The apparatus includes a user interface configured to display rider performance information. The user interface may comprise a display screen, touch screen, indicator panel, mobile-device interface, or other output device. The user interface may also receive user inputs relating to rider identification, session selection, calibration, and operating commands. In one implementation, the user interface displays one or more of:
(a) current cadence, average cadence, and cadence variation;
(b) current torque, average torque, and torque variation;
(c) estimated fatigue level;
(d) predicted future performance;
(e) session progress metrics; and
(f) rider condition metrics.

The apparatus further includes a communications module configured to transmit rider performance data to a remote system. The communications module may communicate by wired or wireless connection, including Wi-Fi, Bluetooth, cellular communication, Ethernet, or other data communication arrangements. A healthcare provider, therapist, physician, trainer, or other authorized user may access transmitted data and may modify operating parameters, controller optimization parameters, safety parameters, or exercise plan parameters remotely. Modified parameters may be incorporated into the exercise planning module and the adaptive rider module.

### Exercise Planning Module

In one implementation, the exercise planning module stores or generates one or more exercise programs for the rider. The exercise programs may include warm-up segments, steady-state segments, interval segments, rehabilitation segments, recovery segments, or endurance segments. The exercise planning module may define target cadence ranges, target torque ranges, segment durations, progression logic, or session objectives. The adaptive rider module may then select operating parameters that implement the exercise program while adapting to the rider's current performance state.

### Overall Operation

During operation, a rider engages the stationary exercise bicycle and actuates the pedal assembly. The sensor subsystem acquires data representative of rider input and apparatus operating state, including cadence, torque, force, and motor current. The control system processes the acquired data and updates the rider-specific dynamic performance model. Based on the rider-specific dynamic performance model and one or more exercise objectives, the adaptive rider module evaluates prospective operating conditions and determines motor operating commands. The motor controller regulates the electric motor to produce the selected torque and/or speed behavior at the pedal assembly. As the rider continues pedaling, dynamic changes in rider-applied force change motor loading. These changes are measured, estimated, or inferred and are incorporated into subsequent control decisions. The control cycle repeats during the session so that motor operation is adapted to the rider's measured and estimated condition.

In this manner, the embodiment provides a motorized stationary exercise bicycle having a motor-driven pedal assembly, a sensor subsystem configured to measure rider interaction, and a control system configured to adapt motor operation based on a rider-specific performance model. rider module.

### Additional Embodiment—Clinical Therapy Implementation for Parkinson's Disease

In another embodiment, a motorized stationary exercise apparatus is configured for therapeutic use with an individual having Parkinson's disease (PD) or one or more Parkinsonian symptoms. In some embodiments, the apparatus incorporates Parkinson's disease-specific sensing, modeling, control, monitoring, and safety functions so that operation of a motor-driven exercise member may be personalized for a rider exhibiting one or more neuromotor symptoms. Optionally, the apparatus may be configured to provide assisted exercise, resisted exercise, guided exercise, or combinations thereof while monitoring rider performance and modifying operation in response to rider-specific symptom manifestations.

In some embodiments, the apparatus comprises a frame supporting a rider support structure and a rider engagement structure. The rider support structure may include a seat, saddle, recumbent support, back support, or other body-supporting component. The rider engagement structure may include handlebars, handgrips, arm supports, or other rider-contacting elements. A motor-driven pedal assembly may be attached to or supported by the frame and may be configured to be actuated by the rider. In some embodiments, the apparatus further includes a sensor system configured to measure pedal speed, pedal torque, cadence, motor contribution, and optionally additional quantities indicative of rider interaction with the motor-driven pedal assembly. A control system and motor controller, which may include one or more processors and one or more memory devices storing executable instructions, may be operatively coupled to the sensor system and to the motor-driven pedal assembly. Optionally, the apparatus further includes a personalized exercise planning module, an adaptive rider module, a clinical interface, and a secure communications module.

The apparatus may be deployed in a neurology clinic, outpatient rehabilitation facility, senior center, wellness center, specialized movement disorder center, hospital-associated therapy program, or home-based supervised therapy environment. Optionally, the apparatus may be used under direct supervision of a clinician, therapist, trainer, or caregiver, or may be used in a remotely monitored setting in which rider performance data are transmitted to an external monitoring system for review and adjustment of therapy parameters.

In another aspect, a method of providing therapy for a rider having Parkinson's disease or Parkinsonian symptoms may include operating a motorized stationary exercise apparatus while measuring rider interaction with a pedal assembly, determining one or more rider-condition variables from measured, estimated, or inferred operating data, and controlling motor operation based at least in part on the rider-condition variables. Optionally, the method may include establishing a rider-specific model, updating the rider-specific model during and/or across sessions, and selecting motor speed commands, motor torque commands, or both, based on one or more therapy objectives and one or more safety criteria.

### Parkinson's-Specific Rider Model

In this implementation, an adaptive rider module constructs and updates a rider-specific dynamic performance model incorporating neuromotor characteristics associated with Parkinson's disease. In some embodiments, the model represents a current and evolving performance state of the rider during operation of the apparatus and may be configured to capture symptom manifestations that affect cycling motion, therapeutic response, rider tolerance, and/or rider safety.

In some embodiments, the rider-specific dynamic performance model includes one or more estimates of tremor amplitude, tremor frequency, cadence variability, rigidity-related resistance, dystonia-related irregularity, force asymmetry, force variability, bradykinesia-related movement slowing, fatigue progression under repetitive motion, movement initiation delay, cycle-to-cycle temporal irregularity, or combinations thereof.

Optionally, one or more of these model parameters may vary as a function of pedal speed, commanded motor torque, rider effort, session duration, medication state, time of day, exercise mode, or combinations thereof.

In some embodiments, sensor data used to estimate the foregoing variables include signals from high-resolution encoders, shaft rotational position sensors, cadence sensors, torque sensors, current sensors, crank angle sensors, force sensors, bilateral or multi-point sensing arrangements, and/or controller-derived estimates. Cadence fluctuations, torque oscillations, temporal asymmetry between successive pedal strokes or cycle portions, transient delays in force development, and oscillatory loading behavior may be extracted from these signals and used by the adaptive rider module to estimate rider condition. Additionally or alternatively, one or more of these quantities may be derived from motor controller operating data, including estimated shaft torque, speed regulation error, motor current variation, or other observed drive characteristics.

In some implementations, the adaptive rider module continuously updates the Parkinson's-specific rider model during a riding session and stores updated parameters for use in later sessions. Thus, the model may capture within-session changes, such as fatigue accumulation, transient symptom fluctuation, medication-related variation, or cadence instability, and cross-session changes, such as rehabilitation progress, symptom progression, adaptation to therapy, or response to a prescribed cycling protocol.

In another aspect, a method may include generating, updating, and storing a rider-specific model for an individual having Parkinson's disease, the model being based on measured rider performance and configured to predict rider response to one or more candidate operating conditions. Optionally, the method may include retrieving the stored model at the beginning of a later session and using the model to initialize therapy parameters for the later session.

### High-Cadence Therapeutic Strategy

In one implementation, the apparatus is configured to support a therapeutic cycling strategy in which a target cadence range and a target resistance, effort, or performance range are prescribed for a rider diagnosed with Parkinson's disease. In some embodiments, a rider performance objective function is configured to promote maintenance of cadence, resistance, power stability, movement regularity, and/or motion quality within one or more prescribed ranges while accounting for fatigue, instability, asymmetry, and symptom-related oscillation.

In some embodiments, the objective function includes weighted components corresponding to one or more of: achieving target cadence values; achieving target power, torque, or variability values; reducing tremor-induced oscillation amplitude; maintaining cadence and resistance within prescribed ranges; controlling fatigue accumulation rate; minimizing asymmetric pedaling force or timing; reducing cadence interruptions; reducing motion irregularity; and/or maintaining a desired therapeutic intensity. Optionally, the weighting of these components may be set by default, may be determined from rider-specific data, may be selected according to a prescribed therapy mode, or may be entered or modified by clinical personnel through a local interface or a remote system.

In some embodiments, the system actively assists the rider to achieve and maintain clinically prescribed cadence and resistance ranges. For example, where the rider is unable to sustain a desired cadence because of bradykinesia, rigidity, fatigue, freezing tendency, or symptom fluctuation, the motor may increase assistive torque to support pedal motion. Conversely, where symptoms are reduced and the rider is capable of greater effort, the control system may reduce assistance, increase resistance, or otherwise modify the operating state so that therapeutic loading remains within a prescribed range.

In another aspect, a therapy method may include establishing one or more cadence targets, resistance targets, symmetry targets, motion-stability targets, or fatigue-related thresholds for a rider having Parkinson's disease, monitoring rider performance relative to the targets, and modifying motor assistance and/or resistance to maintain rider performance within the targets or within threshold ranges associated with the targets.

### Adaptive Model-Based Predictive Control for Parkinson's Disease

In one implementation, adaptive model predictive control (MPC) is used to determine motor operating commands for therapy of a rider diagnosed with Parkinson's disease. At each control interval, the control system may evaluate prospective pedal speed trajectories, pedal torque trajectories, or combined speed-and-torque trajectories over a future time horizon. The control system may predict rider response under each candidate trajectory using the Parkinson's-specific rider model.

In some implementations, the prediction process accounts for estimated tremor dynamics, predicted fatigue progression, rigidity-related resistance changes, bradykinesia-related movement slowing, cadence instability, asymmetry measures, rider-specific response to assistive or resistive torque, and/or one or more medication-related or time-dependent factors. Optionally, the predicted response may be evaluated over a receding horizon so that the controller repeatedly updates its decision based on recent rider measurements.

In another implementation, the controller selects torque commands, speed commands, or both, that achieve desired cadence and power variability targets, reduce tremor-induced variability, maintain therapeutic intensity, and prevent excessive fatigue. The optimization may be subject to constraints such as maximum torque, minimum torque, allowable cadence range, maximum torque slew rate, maximum speed slew rate, fatigue thresholds, asymmetry thresholds, and safety-related operating limits. In this manner, the control system may determine a motor command profile that is therapeutically useful while remaining within rider-specific tolerances.

In another implementation, motor assistance may be increased during periods of rigidity, bradykinesia, freezing tendency, or increasing fatigue to maintain a target cadence. Additionally or alternatively, resistance may be decreased during such periods. In other operating intervals, such as when rider performance is stable and symptoms are reduced, assistive torque may be reduced and resistive torque may be increased to maintain the desired therapy intensity.

In another aspect, a method may include predicting, by a control system, a rider response to a plurality of prospective operating conditions; evaluating a therapy objective function for the prospective operating conditions; selecting one or more operating conditions based at least in part on the evaluated objective function; and controlling a motorized pedal assembly according to the selected operating conditions.

### Tremor Detection and Compensation

In some embodiments, the sensor subsystem is configured to detect oscillatory behavior indicative of tremor. Tremor-related oscillation may appear in torque signals, cadence signals, crank angle signals, motor current variation, motion variability metrics, or other measured or derived quantities. The adaptive rider module may estimate tremor amplitude, tremor frequency, tremor persistence, and/or cycle-phase relationship using signal filtering, time-domain analysis, frequency-domain analysis, time-frequency analysis, multiresolution analysis, or combinations thereof.

In an implementation, the control system analyzes oscillatory torque components superimposed on the rider's nominal pedaling effort. The system may distinguish periodic tremor-related oscillation from intentional pedaling torque, transient disturbance, and other non-tremor signal components by reference to frequency content, phase continuity, amplitude consistency, pedal-cycle synchronization, rider-specific tremor signatures, or combinations thereof. If tremor amplitude exceeds a threshold, or if tremor characteristics satisfy one or more detection criteria, the system may implement a compensatory control action. Such compensatory action may include increasing motor smoothing torque, stabilizing cadence by assistive torque modulation, adjusting resistance to damp oscillatory behavior, reducing abrupt control transitions, modifying the target operating state to reduce instability, or combinations thereof. Optionally, the degree of compensation may vary as a function of tremor severity, rider fatigue, current cadence, medication phase, prior session data, or historical rider response.

In another aspect, a therapy method may include detecting one or more oscillatory components associated with tremor from exercise-apparatus sensor data, estimating one or more tremor characteristics from the oscillatory components, and adjusting motor operation based at least in part on the estimated tremor characteristics.

### Medication Timing Adaptation

In one implementation, the user interface permits entry of medication timing, medication type, medication phase, expected medication onset, recent dose history, or other medication-related information relevant to Parkinson's symptom presentation. In some embodiments, the adaptive rider module incorporates a medication state into the rider-specific performance model so that control decisions are informed by whether the rider is in an anticipated “on” phase, “off” phase, transition period, or other clinically relevant medication-related condition.

In one implementation, the system adjusts therapy intensity depending on medication phase. For example, during an “off” period, increased motor assistance may be provided, cadence targets may be adjusted, resistance may be reduced, and/or near-term performance targets may be lowered so that the rider can continue therapeutic cycling despite worsened symptoms. During an “on” period, resistance and/or near-term performance targets may be increased to provide greater strengthening, endurance challenge, or movement practice when symptoms are reduced. Optionally, medication phase may also affect tremor suppression weighting, fatigue thresholds, asymmetry correction strategy, permissible cadence range, or progression rate.

In another aspect, a method may include receiving medication-related input for a rider, determining a medication-related rider state based at least in part on the medication-related input, and selecting one or more therapy parameters based at least in part on the medication-related rider state.

### Bilateral Monitoring and Asymmetry Management

In some embodiments, force production, torque production, timing, phase relationship, or cycle-to-cycle consistency may be measured or estimated at multiple locations, multiple cycle phases, multiple portions of a pedal revolution, and/or separately for different sides of the pedal assembly. The adaptive rider module may detect asymmetry in rider contribution, timing, phase progression, smoothness, or other movement characteristics, and such asymmetry may be used as an indicator of impairment severity, fatigue state, unilateral weakness, bradykinesia asymmetry, or therapy response.

Where asymmetry exceeds a threshold, the control system may implement one or more compensatory or corrective actions. In some embodiments, the system may adjust assistive torque delivery, resistive torque delivery, phase-specific control, cadence targets, resistance targets, or symmetry-oriented control weights so as to alter relative work demand, improve movement regularity, encourage more symmetric contribution, or facilitate transition through selected portions of the pedal cycle. For example, assistive torque may be decreased in association with one portion of a cycle and/or increased in association with another portion of a cycle to influence relative rider contribution without requiring limb-specific actuation in a narrow sense. In another example, torque may be applied during one cycle phase to facilitate movement into a subsequent cycle phase. Thus, the control system need not be limited to independently actuating a particular limb, but may instead shape pedal dynamics in a manner that tends to promote improved symmetry or continuity of motion.

In some embodiments, asymmetry measures are tracked longitudinally and used by the adaptive rider module and/or clinical personnel to assess rider progression, symptom change, impairment severity, medication-related variability, or response to therapy.

In another aspect, a method may include determining an asymmetry-related measure from sensor data associated with pedaling, comparing the asymmetry-related measure with one or more thresholds or target values, and adjusting motor operation based at least in part on the comparison.

### Safety Protocols for Parkinson's Disease Therapy

In some implementations, the control system is configured to monitor for unsafe or abnormal operating conditions that may be of particular relevance to a rider diagnosed with Parkinson's disease. Such conditions may include abrupt torque drop-offs, sudden cadence irregularity, excessive oscillatory torque behavior, freezing-like motion interruption, abrupt rider disengagement, excessive asymmetry, sustained instability, unexpected cessation of rider contribution, or sensor patterns indicative of loss of controlled pedaling.

Upon detection of an unusual condition, the control system may classify the condition and select an appropriate pre-planned response. In some embodiments, a pre-planned response may include immediate removal of motor power to the pedals, rapid reduction of assistive or resistive torque, transition to a reduced-assistance mode, controlled deceleration, engagement of electronic and/or electromechanical braking (e.g., dynamic braking), issuance of an alert, or combinations thereof. Optionally, the selected response may depend on severity, persistence, classification confidence, rider state, therapy mode, or combinations thereof. Where the rider is detected to disengage unexpectedly, or where the observed operating state is classified as unsafe or uncertain, the system may automatically slow or stop operation. Optionally, the apparatus may generate a local audible or visual alert or tactile (e.g., vibratory) alert and/or transmit a notification to a remote monitoring system, supervising clinician, or caregiver. The control system may also record the detected event for later review and, in some embodiments, may associate the event with contemporaneous rider-model variables or therapy conditions.

In another aspect, a therapy method may include monitoring sensor data for one or more conditions indicative of instability, potential for freezing, disengagement, or unsafe operation; classifying a detected condition; and automatically implementing a protective control action based at least in part on the classified condition.

### Clinical Data Collection and Monitoring

In some implementations, the apparatus records one or more therapy-related metrics during and across sessions. Recorded data may include tremor amplitude trends, cadence stability metrics, power stability metrics, fatigue progression curves, symmetry indices, rider-model parameter evolution, targeted performance values, actual attained performance values, session duration, session timing, medication-related inputs, safety event data, control parameter histories, and/or derived clinical performance indicators.

Recorded data may be stored locally and/or transmitted through the secure communications module to a remote clinical monitoring system. The remote clinical monitoring system may be configured to permit authorized clinical personnel to review rider progress, compare historical sessions, assess adherence, adjust therapy prescriptions, and monitor symptom-related changes over time. Optionally, transmitted data may be encrypted, access-controlled, anonymized, pseudonymized, or otherwise secured.

In consultation with the personalized exercise planning module, clinical personnel may adjust one or more parameters of bike operation, including target cadence ranges, target resistance ranges, tremor suppression weighting, updated model parameters, fatigue tolerance thresholds, session frequency, session duration, session time-of-day, warm-up strategy, ramp-up strategy, steady-state strategy, cool-down strategy, and/or permitted assistance ranges. In some implementations, such updated parameters are integrated by the adaptive rider module into future sessions so that subsequent control decisions reflect revised clinical goals.

In another aspect, a method may include transmitting rider performance data from the motorized stationary exercise apparatus to a remote system, receiving one or more updated therapy parameters from the remote system, and controlling a later exercise session based at least in part on the updated therapy parameters.

### Longitudinal Therapy Tracking

Over repeated sessions, the adaptive rider module may track changes in rider performance metrics and model parameters. In some implementations, longitudinal tracking includes one or more of cadence frequency, cadence consistency, tremor behavior during cycling, tremor behavior following cycling, endurance duration, torque production stability, bilateral or multi-phase symmetry, fatigue tolerance, response to prescribed cadence targets, or combinations thereof.

Longitudinal tracking may provide objective measures of therapy progression and may be used to identify improvement, plateau, regression, medication-related variability, or time-of-day effects. In some embodiments, the rider-specific model is updated based on this longitudinal information so that the apparatus becomes increasingly personalized over time. Optionally, longitudinal trends may be used to modify future exercise prescriptions, control weighting factors, session timing recommendations, or progression schedules.

In another aspect, a method may include storing data from a plurality of therapy sessions, comparing one or more rider-performance measures across the plurality of therapy sessions, and modifying one or more future control parameters based at least in part on the comparison.

### Functional Characteristics of the Parkinson's Disease Embodiment

In an implementation, the Parkinson's disease therapy embodiment may provide one or more of dynamic cadence stabilization, tremor detection and compensation, fatigue-aware intensity control, medication-aware adaptation, asymmetry-aware control, and goal-directed therapy control. Optionally, the relative emphasis placed on these functions may vary according to rider condition, clinician preference, therapy stage, medication phase, or session objective.

Through integration of Parkinson's-specific neuromotor modeling, adaptive control, symptom-responsive assistance, and clinical oversight functions, the apparatus may deliver a personalized cycling therapy session while maintaining rider safety and permitting clinician supervision. In some embodiments, the system provides a framework in which rider measurements, predicted rider response, and prescribed therapy objectives are combined to determine motor control actions for a rider diagnosed with Parkinson's disease. Optionally, the apparatus may be used for therapeutic exercise, supervised rehabilitation, longitudinal assessment, symptom-responsive training, or combinations thereof.

It will be understood that the Parkinson's-specific features described herein may be implemented individually or in any combination, and that any one or more such features may be omitted in particular embodiments. Likewise, features described in connection with an apparatus may also define corresponding methods of use, operation, control, calibration, monitoring, personalization, and therapy delivery, and features described in connection with a method may also be embodied in corresponding apparatus, systems, controllers, instructions stored in non-transitory computer-readable media, or combinations thereof.

It will further be understood that references herein to a pedal, a limb, a side, a stroke, or a cycle portion are provided by way of example to describe representative measurements and control actions and should not be construed as requiring narrowly isolated limb-specific actuation unless expressly recited. In some embodiments, control may instead be applied at the pedal assembly, crankset, drivetrain, motor, or cycle-phase level so as to influence rider contribution, movement regularity, asymmetry, or continuity of motion without requiring independent direct control of a particular anatomical limb.

### Additional Embodiment—Clinic-To-Home Therapy Continuum for Parkinson'S Disease

In another embodiment, a motorized stationary exercise apparatus is configured for therapy of individuals diagnosed with Parkinson's disease (PD) or exhibiting one or more Parkinsonian symptoms. In some embodiments, the apparatus is configured for use across a continuum of care including supervised clinical deployment and residential deployment. The apparatus may thereby support initial assessment, model generation, therapy initiation, longitudinal adaptation, and continued therapy in different care settings while maintaining a common therapeutic framework.

In some embodiments, the apparatus includes a frame, a rider support structure, a rider engagement structure, a motor-driven pedal assembly, a sensor subsystem, a control system, a motor controller, a personalized exercise planning module, an adaptive rider module, a user interface, and a communications module. The rider support structure may include a seat, saddle, recumbent support, back support, or other body-supporting structure. The rider engagement structure may include handlebars, handgrips, forearm supports, or other rider-contacting structures. The motor controller is configured to control an electric motor mechanically coupled to the pedal assembly to provide assistive torque, resistive torque, or combinations thereof.

In some embodiments, the apparatus incorporates Parkinson's disease-specific sensing, modeling, control, safety, and monitoring functions so that operation of the motor-driven pedal assembly may be personalized for a rider exhibiting one or more Parkinsonian neuromotor symptoms. Such functions may include tremor detection, cadence stabilization, fatigue-aware adaptation, asymmetry-aware control, medication-aware adjustment, and local and/or remote clinical oversight.

### Clinical Deployment

In one implementation, the apparatus is deployed in a supervised clinical setting including, for example, a neurology clinic, outpatient rehabilitation facility, hospital-associated therapy program, specialized movement disorder center, senior center, or wellness center. In some embodiments, the clinical deployment is used to initiate therapy, establish a rider-specific baseline, determine initial safety limits, and define initial therapeutic targets.

In some embodiments, an initial clinical assessment session is performed in which the adaptive rider module collects rider-specific data including cadence stability, tremor amplitude, tremor frequency, torque production capacity, fatigue progression rate, asymmetry-related measures, and other motor-performance parameters before, during, and after cycling. Optionally, the clinical assessment may include one or more warm-up phases, steady-state phases, guided cadence phases, perturbation phases, and recovery phases so that rider response may be characterized under multiple operating conditions.

In some embodiments, the adaptive rider module constructs a rider-specific dynamic performance model based at least in part on the collected clinical data. The rider-specific model may include one or more estimates of tremor dynamics, cadence variability, rigidity-related resistance, bradykinesia-related slowing, fatigue progression, force variability, movement continuity, and asymmetry-related measures. Optionally, the rider-specific model may be generated using rider-specific measurements alone or in combination with historical data, population-derived data, prior clinical records, and/or clinician-entered parameters. Additionally, in some embodiments, the personalized exercise planning module uses the rider-specific model to define a therapy session plan for clinical use. Session parameters may include a target cadence range, a target resistance or power range, an assistive range, fatigue thresholds, tremor suppression weighting, safe torque limits, safe speed limits, and incremental change limits. The control system may then operate the motor-driven pedal assembly according to the therapy session plan while continuously updating the rider-specific model based on measured rider response.

In some implementations, the control system is configured to support a therapeutic cycling strategy in which a target cadence range is selected to promote therapeutic benefit for a rider with Parkinson's disease. Where the rider is unable to sustain the target cadence because of bradykinesia, rigidity, fatigue, tremor, freezing tendency, or symptom fluctuation, the motor controller may increase assistive torque, reduce resistance, or otherwise modify operation to support continuity of motion. Conversely, where rider performance is stable, assistance may be reduced and/or resistance may be increased to maintain a desired therapeutic intensity.

In some embodiments, the sensor subsystem continuously measures cadence variability, torque oscillations, rotational irregularity, and/or other signal features associated with tremor or instability. The adaptive rider module may use time-domain analysis, frequency-domain analysis, time-frequency analysis, multiresolution analysis, filtering, or combinations thereof to estimate tremor amplitude, tremor frequency, tremor persistence, and/or tremor-related phase behavior. If tremor amplitude exceeds a threshold or satisfies another compensation condition, the motor controller may implement one or more compensatory actions including smoothing torque, assistive modulation, resistance adjustment, or reduction of abrupt control transitions.

In some implementations, medication timing, medication phase, expected onset, or related medication information may be incorporated into the rider-specific model and/or the personalized exercise planning module. The control system may thereby adapt therapy intensity, cadence targets, assistance levels, resistive levels, fatigue thresholds, and/or tremor compensation strategy according to predicted medication efficacy periods or learned medication-response patterns. Additionally, asymmetry-related measures including pedal torque asymmetry, force asymmetry, timing asymmetry, phase asymmetry, or related movement asymmetry may be monitored during clinical deployment. If asymmetry exceeds a threshold, the control system may implement one or more corrective actions including adjustment of assistive torque, adjustment of resistive torque, modification of cadence targets, modification of resistance targets, phase-specific torque shaping, or changes in objective-function weighting.

In some embodiments, the control system continuously evaluates operating conditions for indications of unsafe or abnormal rider state, including excessive fatigue accumulation, abnormal torque fluctuations, abnormal cadence fluctuations, tremor escalation, freezing-like interruption, sudden disengagement, sudden torque changes, unexpected cessation of rider contribution, or other abnormal operating conditions. If unsafe conditions are detected, the system may reduce torque, reduce resistance, increase assistance, transition to an assistive-only mode, transition to a fail-safe mode, perform controlled deceleration, engage braking, or stop the session. Clinical personnel may review rider performance in real time or after the session and may adjust one or more therapy parameters including session length, session frequency, cadence targets, resistance or power targets, tremor suppression parameters, assistance strategy, torque limits, target improvement trajectory, progression rules, and/or objective-function weightings. The resulting rider-specific model and therapy parameters may be stored and later used in additional clinical sessions, residential sessions, or both.

### Residential Deployment

In a continuum of a clinical deployment, the apparatus is deployed in a residential setting including, for example, a private residence, assisted-living environment, caregiver-supervised residence, or another non-clinical environment. In some embodiments, the residential deployment is configured to continue therapy established in the clinical setting while using the rider-specific model, therapeutic targets, and safety parameters generated or refined through prior sessions.

In some embodiments, the residential deployment includes guided operation and automated calibration features suitable for substantially independent use by the rider. The user interface may provide setup instructions, readiness-assessment prompts, medication-entry prompts, safety reminders, guided warm-up prompts, and session-completion prompts. Optionally, the user interface may be simplified for riders affected by tremor, bradykinesia, rigidity, fatigue, or other motor or cognitive limitations.

In some embodiments, prior to each residential therapy session, the personalized exercise planning module performs a readiness assessment. The readiness assessment may incorporate medication timing, self-reported symptom severity, sleep-related information, perceived fatigue, recent session history, recent safety events, and/or other rider-condition information. Based on the readiness assessment and previously stored rider-specific model data, the system may estimate the rider's current capability and capacity for exercise and may adjust session targets accordingly.

In some embodiments, the personalized exercise planning module generates a residential therapy session plan based on the rider-specific model, the readiness assessment, historical session data, and one or more therapeutic objectives. The rider performance objective function may prioritize neuromotor performance, stabilization, continuity of motion, and therapeutic cadence maintenance. Optionally, the objective function may also account for fatigue accumulation, tremor suppression, asymmetry reduction, safety margins, adherence goals, and/or a target improvement trajectory over time. Residential session parameters may include one or more of a target cadence range, a target resistance or power range, a target assistive range, a fatigue threshold, a tremor suppression weighting, safe torque limits, safe speed limits, and incremental change limits. In some embodiments, the residential deployment uses more conservative limits, earlier intervention thresholds, additional prompts, additional alerts, or reduced permissible operating envelopes as compared with a supervised clinical deployment.

During residential operation, the sensor subsystem continuously measures cadence variability, torque oscillations, rotational irregularity, and/or other signal features associated with tremor or instability. The adaptive rider module estimates tremor amplitude, tremor frequency, tremor persistence, and/or tremor-related phase behavior and may automatically invoke tremor compensation when one or more thresholds are exceeded. Such compensation may include smoothing torque, assistive modulation, resistance adjustment, reduced abruptness in control transitions, or modification of target operation within a prescribed range.

In some embodiments, medication timing entered by the rider is incorporated into the personalized exercise planning module and/or the rider-specific model. The system may adapt therapy intensity, cadence targets, assistive levels, resistive levels, fatigue thresholds, and tremor compensation strategy according to predicted medication efficacy periods or learned medication-response patterns. Over time, the system may learn rider-specific medication response and use such learned response in subsequent residential sessions and later clinical reassessment.

In some embodiments, asymmetry-related measures including pedal torque asymmetry, force asymmetry, timing asymmetry, phase asymmetry, or other movement asymmetry are monitored during residential deployment. If asymmetry exceeds a threshold, the control system may implement one or more corrective actions including adjustment of assistive torque, adjustment of resistive torque, modification of cadence targets, modification. of resistance targets, phase-specific torque shaping, or changes in objective-function weighting. In some embodiments, such control is applied at the pedal assembly, crankset, drivetrain, motor, or cycle-phase level so as to encourage more coordinated or more symmetric rider contribution without requiring narrowly isolated limb-specific actuation.

Because residential operation may occur without continuous in-person clinical supervision, the control system may implement additional safeguards. In some embodiments, the system continuously evaluates excessive fatigue accumulation, abnormal torque fluctuations, abnormal cadence fluctuations, tremor escalation, freezing-like interruption, sudden disengagement, sudden torque changes, unexpected cessation of rider contribution, and/or other abnormal operating conditions. If unsafe conditions are detected, the system may reduce torque, reduce resistance, increase assistance, transition to an assistive-only mode, transition to a fail-safe mode, perform controlled deceleration, engage braking, or stop the session. Audible, tactile (e.g., vibratory) and visual alerts may be provided to the rider, and optionally remote alerts, caregiver alerts, or clinician notifications may also be generated.

In some embodiments, the communications module securely transmits session data, rider data, rider-model data, safety-event data, and/or therapy-summary data from the residential deployment to a clinically accessible remote monitoring platform. Based on analysis of transmitted data and patient history, clinical personnel may remotely adjust one or more therapy parameters including session length, session frequency, cadence targets, resistance or power targets, tremor suppression parameters, assistance strategy, torque limits, target improvement trajectory, progression rules, and/or objective-function weightings.

Updated parameters may be incorporated into future residential sessions and, in some embodiments, into later clinical sessions. Across repeated residential sessions, the adaptive rider module may track longitudinal changes in rider performance and rider-model parameters. In some embodiments, tracked changes include tremor amplitude during cycling, duration of stable high-cadence cycling, achievement of targeted performance improvement over time, symmetry-related measures, fatigue tolerance, required level of torque or speed assistance, cadence stability, and/or aerobic capacity. These longitudinal metrics may be used to update the rider-specific model, support clinician review, modify future therapy plans, identify improvement or regression, and/or adjust target improvement trajectories.

### Coordinated Continuum Operation

In some embodiments, the clinical deployment and the residential deployment share a common rider-specific model and a common control architecture, while differing in session structure, safety constraints, supervision level, user-interface complexity, and permissible operating limits. Thus, measurements obtained in the clinical setting may inform residential session planning, and measurements obtained in the residential setting may inform later clinical reassessment, adjustment, or progression of therapy.

In another aspect, a method of providing Parkinson's disease therapy across a clinic-to-home continuum may include performing an initial rider assessment in a clinical setting or a residential setting, generating a rider-specific dynamic performance model based at least in part on measured rider data, storing the rider-specific model, conducting a later therapy session in a different care setting using the stored rider-specific model, measuring rider interaction with a motor-driven pedal assembly during the later therapy session, updating the rider-specific model based on the measured rider interaction, and controlling motor operation based at least in part on the updated rider-specific model.

In yet another aspect, a system may include a clinically deployed motorized stationary exercise apparatus, a residentially deployed motorized stationary exercise apparatus, and a remote platform configured to exchange rider-specific model information and therapy parameters between the clinically deployed apparatus and the residentially deployed apparatus. Optionally, the clinically deployed apparatus and the residentially deployed apparatus may be the same physical unit used in different deployment modes, or different physical units operating according to a shared rider-specific model and shared therapy architecture.

In another aspect, non-transitory computer-readable media may store instructions that, when executed by one or more processors, cause a motorized stationary exercise system to perform one or more of the assessment, modeling, planning, monitoring, safety, adaptation, transmission, or control functions described herein.

It will be understood that the Parkinson's disease therapy features described herein may be implemented individually or in any combination, and that any one or more such features may be omitted in particular embodiments. Features described in connection with the clinical deployment may also be used in the residential deployment, and features described in connection with the residential deployment may also be used in the clinical deployment.

It will further be understood that features described in connection with an apparatus may also define corresponding methods of use, calibration, readiness assessment, session planning, control, monitoring, safety response, remote supervision, and therapy delivery, and that features described in connection with a method may also be embodied in corresponding apparatus, systems, controllers, user interfaces, remote platforms, or instructions stored in non-transitory computer-readable media. Additionally, it will further be understood that references herein to bilateral symmetry, left-right balance, phase-specific torque shaping, differentiated rider contribution, or related concepts are provided by way of example to describe representative sensing and control strategies and should not be construed as requiring narrowly isolated limb-specific actuation unless expressly recited. In some embodiments, control may instead be applied at the pedal assembly, crankset, drivetrain, motor, or cycle-phase level so as to influence rider contribution, coordination, symmetry, cadence stability, or continuity of motion without requiring direct independent control of a particular anatomical limb.

### Additional Embodiment—Neuromotor Entrainment Implementation for Parkinson'S Disease

In another embodiment, a motorized stationary exercise apparatus is configured as a neuromotor entrainment therapy platform for individuals diagnosed with Parkinson's disease (PD) or another neurodegenerative, neurologic, or neuromotor disorder. In some embodiments, this embodiment emphasizes rhythmic motor entrainment, cadence synchronization, phase stabilization, and adaptive neural stimulation through controlled motor-driven cycling. Optionally, the apparatus may be configured to address rider conditions associated with abnormal motor rhythm generation, impaired internal cueing, excessive oscillatory activity, bradykinesia, rigidity, tremor, freezing tendency, impaired bilateral coordination, or combinations thereof. Additionally or alternatively, the apparatus may be configured to deliver a non-invasive therapy in which externally imposed rhythmic structure is used to influence rider movement patterns and, optionally, longer-term neuromotor adaptation.

In some embodiments, this embodiment is based in part on the recognition that certain riders diagnosed with Parkinson's disease or other neurologic disorders may exhibit abnormal rhythmicity, oscillatory irregularity, reduced movement automaticity, impaired internal timing, impaired sensory-motor coupling, or diminished spontaneous movement synchronization. In some embodiments, externally supplied rhythmic input, cadence stabilization, torque shaping, sensory cueing, or combinations thereof may promote synchronization between rider motor output and a prescribed therapeutic rhythm. Optionally, the apparatus may be configured to use controlled pedal motion, controlled torque delivery, and/or synchronized multisensory cues to facilitate entrainment of rider movement to a target rhythm. Additionally or alternatively, the apparatus may be configured to reduce movement irregularity, reduce tremor-related variability, improve continuity of pedaling, promote bilateral coordination, and/or improve sustained cadence stability.

### Neuromotor Entrainment Configuration

In one embodiment, the invention comprises a motorized stationary exercise apparatus implemented as a stationary exercise bicycle having a motor-driven pedal assembly, a personalized exercise planning module, and an adaptive rider module configured to dynamically personalize exercise operation for an individual rider. In some embodiments, the personalized exercise operation is used by a motor controller to dynamically control the speed and torque of an electric motor mechanically coupled to the pedal assembly. Changing rider foot force on the pedal assembly may inject dynamic load changes into motor operation, and such dynamic load changes may be detected, estimated, or inferred by the motor controller and analyzed by the control system. Optionally, the control system may use such dynamic load changes to characterize rider engagement, rhythm stability, fatigue progression, symptom expression, or entrainment response.

In some embodiments, the control system is configured to induce rhythmic entrainment of lower-limb motor patterns through controlled cadence modulation, assistive torque shaping, phase-specific torque shaping, resistive shaping, or combinations thereof. Optionally, the control system may impose, reinforce, or stabilize a target therapeutic rhythm by introducing periodic or quasi-periodic control inputs into the motor-driven pedal assembly. Additionally or alternatively, the system may guide the rider toward synchronization with a target cadence without requiring abrupt forced motion, and may instead gradually influence rider timing, phase relationship, or movement regularity over time.

In some embodiments, the apparatus includes a frame, a rider support structure, a rider engagement structure, a motor-driven pedal assembly, a sensor subsystem, a control system, a motor controller, a personalized exercise planning module, an adaptive rider module, a user interface, and, optionally, a communications module. The rider support structure may include a seat, saddle, recumbent support, back support, or another body-supporting structure. The rider engagement structure may include handlebars, handgrips, forearm supports, armrests, or other rider-contacting structures. In some embodiments, the motor controller is configured to regulate assistive torque, resistive torque, cadence, phase-specific torque shaping, or combinations thereof according to one or more entrainment-oriented control objectives.

### Neural Rhythm Modeling

In some embodiments, the personalized exercise planning module and the adaptive rider module collaborate to construct a neuromotor rhythm model representing intrinsic cycling rhythm characteristics of the rider. The neuromotor rhythm model may represent one or more rider-specific rhythmic features that are relevant to Parkinson's disease therapy, rhythm synchronization, movement regularity, or neuromotor entrainment. Optionally, the neuromotor rhythm model may be used to define a baseline rider rhythm, identify deviations from rhythmic stability, estimate response to external rhythmic cueing, and determine future motor control actions intended to improve synchronization or reduce oscillatory irregularity.

In some embodiments, the neuromotor rhythm model estimates one or more of: baseline cadence frequency; cadence variability; torque variability; power variability; cadence and torque/power variability spectrum; tremor-related oscillatory components; phase lag between successive pedal strokes; phase lag between left and right pedal strokes; phase drift over time; cadence coherence; symmetry-related timing measures; freeze-related interruptions; and/or rider responsiveness to externally imposed rhythmic inputs. Additionally or alternatively, the neuromotor rhythm model may include measures of phase stability, entrainment susceptibility, phase-reset behavior, or rider-specific sensitivity to cadence perturbation.

In some embodiments, the neuromotor rhythm model includes temporal-domain, frequency-domain, and/or time-frequency-domain representations derived from multiresolution analysis, filtering, spectral estimation, coherence analysis, phase analysis, or combinations thereof applied to cadence, torque, power, crank-angle, and/or motor-current signals. Optionally, one or more model components may be generated using wavelet analysis, bandpass filtering, short-time Fourier analysis, phase-locking estimation, or other rhythmic-signal characterization techniques. Additionally or alternatively, model parameters may be updated continuously, periodically, or event-driven during a riding session.

In some embodiments, the adaptive rider module updates the neuromotor rhythm model continuously so as to reflect disease-state fluctuations, medication cycles, fatigue progression, rider adaptation, or changing response to rhythmic cueing. Optionally, the neuromotor rhythm model may be updated within a session and also across multiple sessions, thereby allowing the system to track both transient state changes and longer-term therapeutic progression. Additionally or alternatively, the neuromotor rhythm model may be stored locally, transmitted to a remote clinical platform, or used by the personalized exercise planning module to define later therapy targets.

### Rhythmic Cue Integration

In some embodiments, the apparatus provides rhythmic entrainment cues through controlled motor torque pulses, subtle cadence modulation, phase-specific assistive inputs, phase-specific torque shaping, resistive shaping, or combinations thereof. Optionally, the control system may introduce low-amplitude periodic torque inputs synchronized to a target cadence, may gradually shift cadence phase to entrain rider pedaling rhythm, and/or may provide tempo-stabilizing torque assistance. Additionally or alternatively, the control system may impose a stable cadence scaffold while allowing the rider to remain actively engaged in the movement.

In some embodiments, rhythmic inputs are selected so as to encourage synchronization between rider motor output and a prescribed therapeutic cadence without imposing excessive abruptness or discomfort. Optionally, such inputs may be periodic, quasi-periodic, intermittent, phase-targeted, amplitude-limited, or progressively varying over time. Additionally or alternatively, cue amplitude, pulse width, phase timing, cue frequency, or phase-specific torque shaping may be adjusted as a function of rider responsiveness, fatigue state, symptom severity, medication state, cadence stability, or safety constraints.

In some embodiments, the control system may gradually alter cadence phase or cadence setpoint in order to entrain rider movement to a new target cadence. Optionally, cadence changes may be introduced incrementally over multiple pedal revolutions, over one or more session segments, or over repeated therapy sessions. Additionally or alternatively, the target cadence may be advanced, delayed, held constant, or adaptively selected based on rider-specific entrainment response.

### Phase Synchronization Control

In some embodiments, the pedal assembly includes one or more angular position sensors, encoders, crank-angle sensors, power-meter-derived phase sensors, or other sensing arrangements allowing real-time measurement or estimation of phase difference between successive pedal strokes, between opposite sides of the pedal assembly, or between rider effort and an externally imposed target rhythm. Optionally, phase-related information may be obtained from a dedicated sensor subsystem, from a cycling power meter, from a power-phase analysis technique, from crank-angle estimation, or from controller-derived measurements.

In some embodiments, the adaptive rider module evaluates phase synchronization between left and right pedal strokes and calculates a phase coherence metric. Such a phase coherence metric may represent a degree of synchronization, phase consistency, phase stability, phase-locking tendency, or related rhythmic coupling behavior over time. Optionally, the phase coherence metric may vary along a continuum from relatively independent movement to relatively synchronized movement. Additionally or alternatively, the system may determine one or more other synchronization measures, including phase-lock value, phase error, phase drift rate, cycle-to-cycle phase deviation, or symmetry-related timing deviation. If phase deviation exceeds a threshold, or if a synchronization measure falls outside a target range, the control system may implement one or more corrective actions.

Such actions may include, optionally, selective assistive torque to restore synchronization, cadence modulation to re-establish phase alignment, phase-specific torque shaping, resistance adjustment, modification of cue timing, or combinations thereof. Additionally or alternatively, the control system may reduce phase deviation gradually over successive cycles so as to avoid abrupt rider perturbation. In some embodiments, such control promotes bilateral neuromotor coordination, improved continuity of pedaling, and/or improved rhythmic consistency.

### Medication-State-Adaptive Therapy

In some embodiments, the system tracks medication cycles and learns rider-specific response patterns to dopaminergic medication, adjunct medication, or other therapies. The personalized exercise planning module may estimate motor responsiveness as a function of medication based on cadence stability during prior sessions, tremor amplitude trends, phase-coherence trends, fatigue tolerance, or other rider-performance measures. This information may then be used to determine one or more operating targets for a subsequent exercise session, and such information may be passed to the adaptive rider module for real-time implementation.

In some embodiments, the system may increase therapeutic intensity during optimal medication windows and may prioritize stabilization strategies during reduced medication efficacy periods. Optionally, the system may increase cadence targets, reduce assistive torque, increase resistance, or increase entrainment challenge during periods associated with improved rider responsiveness. Additionally or alternatively, the system may increase assistance, reduce resistance, reduce cadence-change rate, increase smoothing, increase phase-specific torque shaping, or emphasize synchronization support during periods associated with diminished medication efficacy, increased tremor, or reduced movement automaticity.

In some embodiments, the system develops a rider-specific medication-response profile over time and uses that profile to predict likely rider performance for a planned session. Optionally, medication-state adaptation may be based on rider-entered medication timing, clinician-entered data, remotely provided information, or inferred medication-response patterns derived from prior performance history.

### Progressive Neuroplasticity Optimization

In one embodiment, the rider performance objective function emphasizes neuroplastic adaptation, entrainment stability, and movement regularity rather than purely immediate motor-performance improvement. In some embodiments, the rider performance objective function includes one or more optimization components corresponding to reduction in cadence variability over time, decrease in tremor spectral power, increase in sustained synchronized high-cadence duration, increase in phase coherence, reduction in freeze-related interruptions, improvement in movement continuity, and/or reduction in excessive oscillatory content.

Optionally, tuned acoustic input, tactile (e.g., vibratory) input, visual input, or combinations thereof may be used to enhance neuromotor entrainment. For example, music, metronomic audio, rhythmic tones, seat vibration, handlebar vibration, tactile pulses, visual pacing signals, and/or video-display cues may be synchronized to cadence, may lead cadence, may lag cadence, or may otherwise be coordinated with pedal motion. Additionally or alternatively, multisensory cues may be aligned with motor torque pulses or phase-specific torque shaping so that the rider receives a combined rhythmic input across more than one sensory modality.

In some embodiments, the adaptive rider module progressively increases therapeutic cadence targets, resistance targets, synchronization demands, or entrainment challenge when neuromotor stability improves. Optionally, progression may occur within a session, across sessions, or both. Additionally or alternatively, progression may be constrained by fatigue thresholds, tremor thresholds, medication state, safety limits, clinician-entered parameters, or adherence-related criteria. In some embodiments, progression may be determined by the personalized exercise planning module and implemented in real time by the adaptive rider module.

### Clinical Data and Outcome Metrics

In some embodiments, the apparatus records one or more disease-relevant and entrainment-relevant metrics over time. Such metrics may include, optionally, tremor spectral density, tremor amplitude trends, cadence variability indices, power variability indices, bilateral phase coherence metrics, entrainment stability measures, phase error measures, synchronization duration measures, freeze-free cycling duration, cadence consistency, torque stability, fatigue progression measures, and/or rider-response measures associated with externally imposed rhythmic cueing.

In some embodiments, such data may be displayed through a local or remote clinical dashboard and may be transmitted to a remote monitoring system for review by clinical personnel including a therapist, neurologist, physician, or other authorized user. Optionally, such data may be used to prescribe changes in target cadence frequency, resistance, entrainment pulse amplitude, cue timing, phase-specific torque shaping, synchronization difficulty, neuroplastic progression rate, or other therapy parameters. Additionally or alternatively, such data may be used to evaluate rider adherence, progression, stability, or medication-response trends over time.

### Safety Controls

Because this embodiment may involve active cadence changes, active torque modulation, entrainment pulses, phase-specific torque shaping, resistive shaping, or multisensory cueing, the system may include one or more safety safeguards. In some embodiments, such safeguards include maximum cadence limits, torque ceiling thresholds, abrupt disengagement detection, excessive oscillation detection, excessive fatigue detection, cardiovascular monitoring integration, and/or symptom-escalation detection. Optionally, one or more rider-specific safety limits may be established during an initial assessment or updated based on longitudinal rider data.

In some embodiments, if unsafe conditions are detected, the system transitions to an assistive-only mode, a reduced-intensity mode, a stabilization mode, or a safe-stop mode. Additionally or alternatively, the control system may reduce cue amplitude, reduce cadence-change rate, reduce resistance, increase smoothing, reduce phase-specific torque shaping, request rider confirmation, provide visual, tactile (e.g., vibratory) or audible alerts, or notify a caregiver or clinician. Optionally, safety responses may differ according to whether the apparatus is operating in a clinical setting, a residential setting, or another supervised or unsupervised setting.

### Longitudinal Neuromotor Adaptation

Across repeated therapy sessions, the personalized exercise planning module and the adaptive rider module may track one or more longitudinal neuromotor adaptation measures. In some embodiments, such measures include decreases in tremor amplitude before, during, and after cycling; improved phase synchronization; increased freeze-free cycling duration; improved cadence consistency; improved resistance consistency; improved entrainment stability; improved sustained synchronization duration; reduced cue dependence; and/or improved rider tolerance for therapeutic cadence.

In some embodiments, such longitudinal tracking provides objective measures of neuromotor improvement specific to Parkinson's disease or related disorders as a function of phase synchronization, cadence entrainment, movement regularity, and neuromotor stability over time. Optionally, the system may use such longitudinal information to update the rider-specific neuromotor rhythm model, modify future progression rates, revise cueing strategies, or recommend changes in therapeutic targets. Additionally or alternatively, such longitudinal information may be incorporated by the personalized exercise planning module into later session planning and by the adaptive rider module into later real-time control.

### Apparatus, Method, and System Aspects

In another aspect, a method of providing neuromotor entrainment therapy for a rider diagnosed with Parkinson's disease may include measuring rider interaction with a motor-driven pedal assembly, generating a neuromotor rhythm model based at least in part on cadence-related and torque-related rider data, determining one or more synchronization-related metrics, and controlling motor speed, motor torque, cadence modulation, phase-specific torque shaping, or cue timing based at least in part on the neuromotor rhythm model and the synchronization-related metrics. Optionally, the method may further include updating the neuromotor rhythm model during the session and/or across multiple sessions.

In another aspect, a method may include applying one or more rhythmic motor cues to a rider through a motor-driven pedal assembly, detecting rider synchronization response to the one or more rhythmic motor cues, determining whether phase deviation, cadence variability, tremor amplitude, or another rider-performance measure satisfies a threshold, and adjusting one or more therapy parameters in response thereto. Additionally or alternatively, the method may include coordinating one or more non-motor sensory cues with pedal motion, cadence modulation, or phase-specific torque shaping.

In another aspect, a system may include a motorized stationary exercise apparatus, a local or remote clinical dashboard, and one or more processors configured to generate and update a rider-specific neuromotor rhythm model, determine one or more entrainment-related measures, and control motor-driven cycling according to one or more neuromotor entrainment objectives. Optionally, the system may further include a remote platform configured to receive rider data, store longitudinal metrics, and permit clinician modification of entrainment-related therapy parameters, including parameters associated with the rider performance objective function.

In yet another aspect, non-transitory computer-readable media may store instructions that, when executed by one or more processors, cause the motorized stationary exercise apparatus or related system to perform one or more of the sensing, modeling, synchronization analysis, cue generation, adaptation, safety, transmission, or control functions described herein.

### Functional Characteristics

In some embodiments, this embodiment provides one or more of closed-loop neuromotor entrainment, rhythmic motor stabilization, medication-aware adaptation, progressive neuroplastic optimization, and objective disease-specific metrics. Optionally, the relative emphasis placed on one or more such functions may vary according to rider condition, diagnosis, therapy stage, medication state, supervision level, or session objective.

Through integration of rhythm modeling, phase analysis, cadence synchronization, dynamic torque modulation, and optionally multisensory cueing, this embodiment may provide a non-invasive Parkinson's disease-specific or neurologic-disease-specific therapy platform designed to enhance motor function while maintaining rider safety. Additionally or alternatively, the platform may be used in a clinical setting, a residential setting, or across a clinic-to-home therapy continuum.

It will be understood that the neuromotor entrainment features described herein may be implemented individually or in any combination, and that any one or more such features may be omitted in particular embodiments. Features described in connection with cadence modulation may also be used in connection with phase-specific torque shaping, features described in connection with motor cueing may also be used in connection with acoustic, visual, or tactile (e.g., vibratory) cueing, and features described in connection with Parkinson's disease may also be used in connection with other neurologic or neurodegenerative disorders unless expressly stated otherwise.

It will further be understood that features described in connection with an apparatus may also define corresponding methods of use, calibration, rhythm assessment, synchronization analysis, therapy planning, control, monitoring, safety response, and therapy delivery, and that features described in connection with a method may also be embodied in corresponding apparatus, systems, controllers, clinical dashboards, remote platforms, or instructions stored in non-transitory computer-readable media.

It will further be understood that references herein to left and right pedal strokes, bilateral phase synchronization, limb timing, or related concepts are provided by way of example to describe representative sensing and control strategies and should not be construed as requiring narrowly isolated limb-specific actuation unless expressly recited. In some embodiments, control may instead be applied at the pedal assembly, crankset, drivetrain, motor, or cycle-phase level so as to influence rider contribution, synchronization, cadence stability, symmetry, or continuity of motion without requiring direct independent control of a particular anatomical limb.

Although the invention has been shown and described with respect to certain illustrated aspects, it will be appreciated that equivalent alterations and modifications will occur to others skilled in the art upon the reading and understanding of this specification and the annexed drawings. In particular regard to the various functions performed by the above described components (assemblies, devices, circuits, systems, etc.), the terms (including a reference to a “means”) used to describe such components are intended to correspond, unless otherwise indicated, to any component which performs the specified function of the described component (e.g., that is functionally equivalent), even though not structurally equivalent to the disclosed structure, which performs the function in the herein illustrated exemplary aspects of the invention. In this regard, it will also be recognized that the invention includes a system as well as a computer-readable medium having computer-executable instructions for performing the acts and/or events of the various methods of the invention.

The various techniques described herein may be implemented in connection with hardware or software or, where appropriate, with a combination of both. As used in this application, the terms “component”, “module”, “object”, “service”, “model”, “representation”, “system”, “interface”, or the like are generally intended to refer to a computer-related entity, either hardware, a combination of hardware and software, software, or software in execution. For example, a component can be, but is not limited to being, a process running on a processor, a processor, a hard disk drive, a multiple storage drive (of optical and/or magnetic storage medium), an object, an executable, a thread of execution, a program, and/or a computer. By way of illustration, both an application running on a controller and the controller can be a component. One or more components can reside within a process and/or thread of execution and a component can be localized on one computer and/or distributed between two or more computers, industrial controllers, or modules communicating therewith. As another example, an interface can include I/O components as well as associated processor, application, and/or API components.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it can be noted that one or more components may be combined into a single component providing aggregate functionality or divided into several separate sub-components, and that any one or more middle layers, such as a management layer, may be provided to communicatively couple to such sub-components in order to provide integrated functionality. Any components described herein may also interact with one or more other components not specifically described herein but generally known by those of skill in the art.

In addition to the various embodiments described herein, it is to be understood that other similar embodiments can be used or modifications and additions can be made to the described embodiment(s) for performing the same or equivalent function of the corresponding embodiment(s) without deviating there from. Still further, multiple processing chips or multiple devices can share the performance of one or more functions described herein, and similarly, storage can be affected across a plurality of devices. Accordingly, the invention should not be limited to any single embodiment, but rather should be construed in breadth, spirit and scope in accordance with the appended claims.

The subject matter as described above includes various exemplary aspects. However, it should be appreciated that it is not possible to describe every conceivable component or methodology for purposes of describing these aspects. One of ordinary skill in the art may recognize that further combinations or permutations may be possible. Various methodologies or architectures may be employed to implement the subject invention, modifications, variations, or equivalents thereof. Accordingly, all such implementations of the aspects described herein are intended to embrace the scope and spirit of subject claims.

The word “exemplary” is used herein to mean serving as an example, instance, or illustration. For the avoidance of doubt, the subject matter disclosed herein is not limited by such examples. In addition, any aspect or design described herein as “exemplary” is not necessarily to be construed as preferred or advantageous over other aspects or designs, nor is it meant to preclude equivalent exemplary structures and techniques known to those of ordinary skill in the art.

To the extent that the term “includes” is used in either the detailed description or the claims, such term is intended to be inclusive in a manner similar to the term “comprising” as “comprising” is interpreted when employed as a transitional word in a claim. Furthermore, the term “or” as used in either the detailed description or the claims is intended to mean an inclusive “or” rather than an exclusive “or”. That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

What is claimed is:

1. A motorized stationary exercise apparatus, comprising:
a frame;
a rider support assembly supported by the frame;
a pedal assembly rotationally supported by the frame;
an electric motor mechanically coupled to the pedal assembly such that operation of the electric motor applies rotational torque to the pedal assembly;
a sensor subsystem configured to generate sensor signals representative of rider interaction with the pedal assembly;
a motor controller configured to provide electrical drive signals to the electric motor;
a personalized exercise planning module;
a communication interface; and
a control system comprising at least one processor and memory storing executable instructions, the control system being operatively coupled to the sensor subsystem, the motor controller, the personalized exercise planning module, and the communication interface,
the control system being configured to:
receive the sensor signals from the sensor subsystem;
generate and update a rider-specific adaptive rider model based on the sensor signals;
predict, using the adaptive rider model, rider performance under a plurality of prospective pedal operating conditions;
determine target motor operating parameters based on the predicted rider performance to achieve at least one target rider outcome comprising target cadence stability, target endurance duration, target fatigue rate, target symmetry, or target synchronized high-cadence duration; and
cause the motor controller to provide the electrical drive signals to the electric motor to alter operation of the pedal assembly according to the target motor operating parameters during an exercise session.

2. The motorized stationary exercise apparatus of claim 1, wherein the communication interface is configured to transmit rider-performance data to a remote monitoring system accessible by a therapist, physician, trainer, or fitness expert.

3. The motorized stationary exercise apparatus of claim 2, wherein the communication interface is further configured to receive, from the remote monitoring system, modified exercise parameters comprising at least one of a cadence target, a resistance target, a torque target, an endurance target, a fatigue-rate target, a symmetry target, a synchronized high-cadence target, an objective-function weighting, or a progression parameter.

4. The motorized stationary exercise apparatus of claim 3, wherein the control system is configured to determine the target motor operating parameters further based on the modified exercise parameters received through the communication interface.

5. The motorized stationary exercise apparatus of claim 1, wherein the personalized exercise planning module is configured to select the at least one target rider outcome based on historical rider data, rider-specific data, or both.

6. The motorized stationary exercise apparatus of claim 1, wherein the adaptive rider model is updated during the exercise session based on measured rider interaction with the pedal assembly.

7. The motorized stationary exercise apparatus of claim 1, wherein the adaptive rider model is further updated across a plurality of exercise sessions based on stored rider-performance data.

8. The motorized stationary exercise apparatus of claim 1, wherein the predicted rider performance comprises a predicted future mechanical performance state of a rider under the plurality of prospective pedal operating conditions.

9. The motorized stationary exercise apparatus of claim 1, wherein determining the target motor operating parameters comprises evaluating a rider performance objective function corresponding to the at least one target rider outcome.

10. The motorized stationary exercise apparatus of claim 9, wherein the rider performance objective function comprises weighted components corresponding to at least two of cadence stability, endurance duration, fatigue rate, symmetry, and synchronized high-cadence duration.

11. The motorized stationary exercise apparatus of claim 1, wherein determining the target motor operating parameters comprises executing model predictive control that evaluates a plurality of candidate future pedal speed and pedal torque trajectories and selects a trajectory predicted to improve at least one of cadence stability, endurance duration, fatigue rate, symmetry, or synchronized high-cadence duration.

12. The motorized stationary exercise apparatus of claim 11, wherein the model predictive control uses the adaptive rider model to predict rider response over a future time horizon.

13. The motorized stationary exercise apparatus of claim 1, wherein the adaptive rider model includes at least one latent rider state variable representing fatigue, endurance capacity, neuromotor stability, or a combination thereof.

14. The motorized stationary exercise apparatus of claim 13, wherein the control system is configured to update the at least one latent rider state variable based on deviation between predicted rider output and measured rider output.

15. The motorized stationary exercise apparatus of claim 1, wherein the control system is configured to alter at least one of pedal rotational speed, applied pedal torque, cadence trajectory, or phase-specific torque shaping during the exercise session based on the target motor operating parameters.

16. A method of operating a motorized stationary exercise apparatus having a pedal assembly mechanically coupled to an electric motor, the method comprising:
measuring, with a sensor subsystem, rider interaction with the pedal assembly;
processing sensor signals representing the rider interaction;
generating and updating, by at least one processor, a rider-specific adaptive rider model based on the sensor signals;
predicting, by the at least one processor using the adaptive rider model, rider performance under a plurality of prospective pedal operating conditions;
determining target motor operating parameters based on the predicted rider performance to achieve at least one target rider outcome comprising target cadence stability, target endurance duration, target fatigue rate, target symmetry, or target synchronized high-cadence duration;
causing a motor controller to apply electrical drive signals to the electric motor according to the target motor operating parameters to alter operation of the pedal assembly during an exercise session;
transmitting rider-performance data to a remote monitoring system accessible by a therapist, physician, trainer, or fitness expert; and
receiving, from the remote monitoring system, modified exercise parameters for use in a later exercise session or in a subsequent portion of the exercise session.

17. The method of claim 16, wherein determining the target motor operating parameters comprises executing model predictive control that evaluates a plurality of candidate future pedal speed and pedal torque trajectories and selects a trajectory predicted to improve at least one of cadence stability, endurance duration, fatigue rate, symmetry, or synchronized high-cadence duration.

18. A non-transitory computer-readable medium storing instructions that, when executed by at least one processor of a motorized stationary exercise apparatus having a motor-driven pedal assembly, cause the at least one processor to:
receive sensor signals representing rider interaction with the pedal assembly;
generate and update a rider-specific adaptive rider model based on the sensor signals;
predict, using the adaptive rider model, rider performance under a plurality of prospective pedal operating conditions;
determine target motor operating parameters based on the predicted rider performance to achieve at least one target rider outcome comprising target cadence stability, target endurance duration, target fatigue rate, target symmetry, or target synchronized high-cadence duration;
cause a motor controller to apply electrical drive signals to an electric motor mechanically coupled to the pedal assembly according to the target motor operating parameters to alter operation of the pedal assembly during an exercise session;
transmit rider-performance data to a remote monitoring system accessible by a therapist, physician, trainer, or fitness expert; and
receive, from the remote monitoring system, modified exercise parameters for use in a later exercise session or in a subsequent portion of the exercise session.

19. A motorized stationary exercise apparatus configured for neuromotor entrainment, comprising:
a frame;
a pedal assembly rotationally supported by the frame;
an electric motor mechanically coupled to the pedal assembly;
a sensor subsystem configured to generate sensor signals representing rider pedaling;
a personalized exercise planning module;
an adaptive rider module implemented by at least one processor; and
a control system operatively coupled to the sensor subsystem and the electric motor, the adaptive rider module being configured to generate and update a rider-specific neuromotor rhythm model based on the sensor signals, and
the control system being configured to generate electrical drive signals that modulate torque applied by the electric motor in a rhythmic pattern based on the rider-specific neuromotor rhythm model to physically influence pedal rotation and rider neuromotor output.

20. The motorized stationary exercise apparatus of claim 19, wherein the control system is further configured to determine a synchronization-related measure from the sensor signals and to modify at least one of torque modulation timing, torque modulation amplitude, cadence target, or phase-specific torque shaping to increase synchronization of rider pedaling with a prescribed therapeutic rhythm.
